(19) United States
(12) Patent Application Publication
(10) Pub. No.: US 2024/0019424 A1
(43) Pub. Date: Jan. 18, 2024

(54) METHOD FOR RESOLVING COMPLEX, MULTISTEP ANTIBODY INTERACTIONS

(71) Applicant: Hoffmann-La Roche Inc., Little Falls, NJ (US)

(72) Inventors: Tony Christopeit, Schliersee (DE); Tilman Schlothauer, Penzberg (DE)

(73) Assignee: Hoffmann-La Roche Inc., Little Falls, NJ (US)

(21) Appl. No.: 18/451,352

(22) Filed: Aug. 17, 2023

Related U.S. Application Data
(63) Continuation of application No. PCT/EP2022/053569, filed on Feb. 15, 2022.

(30) Foreign Application Priority Data
Feb. 18, 2021 (EP) 21157850.5

(51) Int. Cl. G01N 33/543; G01N 33/68

(52) U.S. Cl. CPC G01N 33/54373; G01N 33/6854; G01N 2333/70535

(57) ABSTRACT

Herein is reported a method for determining antibody-FcRn-interaction comprising the steps of immobilizing FcRn on a solid surface, which is suitable for surface plasmon resonance measurement, individually applying to the solid surface obtained in step a) solutions comprising the antibody at different concentrations and determining the association rate constant and the dissociation rate constant for each concentration, and determining with the rates obtained in step b) the KD-value of the antibody-FcRn-interaction, wherein the immobilized FcRn is monomeric FcRn, the monomeric FcRn is immobilized using functional (capture) groups that are directly attached to said solid surface, the solid surface is free of branched glucan, and the immobilization is at a pH value of from pH 7 to pH 8.

The current invention is in the field of antibody characterization. In more detail, herein is provided a new method for the characterization of antibody-FcRn interaction using surface plasmon resonance and taking into account coating density and type of interaction. With this new method an improved determination of Fc-region as well as antibody affinities to FcRn is provided.

### BACKGROUND OF THE INVENTION

IgG half-life is mediated by a cellular recycling mechanism that relies on pH dependent binding to FcRn. While it is well established that the core interaction site for FcRn is located at the CH2-CH3 elbow region, intriguing new data strongly suggest that also the Fab arms contribute to receptor binding. In theory, an IgG molecule then has multiple FcRn binding sites. Experimental data also support that amino acid variations within the variable domains of IgG antibodies can greatly modulate cellular transport, FcRn binding and half-life. Thus, there is a need to fully understand the complex multistep stoichiometry of the IgG-FcRn interaction.

SPR (surface plasmon resonance) is a biosensor-based technology to measure real time protein-protein interaction. SPR technology has become a standard tool in biopharmaceutical research and development (see, e.g., M. A. Cooper, Nat. Rev. Drug Dis. 1(2002) 515-528; D. G. Myszka, J. Mol. Recognit. 12 (1999) 390-408; R. L. Rich and D. G. Myszka, J. Mol. Recognit. 13 (2000) 388-407; D. G. Myszka and R. L. Rich, Pharm. Sci. Technol. Today 3 (2000) 310-317; R. Karlsson and A. Faelt, J. Immunol. Meth. 200 (1997) 121-133), and is commonly employed to determine rate constants for macromolecular interactions. The ability to determine association and dissociation kinetics for molecular interactions provides detailed insights into the mechanism of complex formation (see, e.g., T. A. Morton, D. G. Myszka, Meth. Enzymol. 295 (1998) 268-294). This information is becoming an essential part of the selection and optimization process for monoclonal antibodies and other biopharmaceutical products (see, e.g., K. Nagata and H. Handa, in Real-time analysis of biomolecular interactions, Springer, 2000; R. L. Rich and D. G. Myszka, Curr. Opin. Biotechnol. 11 (2000) 54-61; A. C. Malmborg and C. A. Borrebaeck, J. Immunol. Meth. 183 (1995) 7-13; W. Huber and F. Mueller, Curr. Pharm. Des. 12 (2006) 3999-4021). In addition, SPR technology allows the determination of the binding activity (binding capacity) of e.g. an antibody binding a target.

Since more than a decade Surface Plasmon resonance (SPR) has been used to investigate antibody-antigen or antibody-receptor interactions, e.g. to determine the pH dependent binding affinity of an antibody to the human Fc receptor neonatal (FcRn) to understand its contribution to antibody recycling efficiency. In view of the complexity of both binding partners, it is impossible by definition to establish an SPR interaction assay that can be evaluated applying a single 1:1 Langmuir kinetic fit.

A variety of strategies have been used to modulate serum persistence of therapeutic proteins and endogenous antibodies by taking advantage of the FcRn salvage pathway, including FcRn enhancing/abrogating mutations, Fc-fusion proteins and competitive inhibition of FcRn binding. However, therapeutic IgGs could have very different half-life that seem to be not correlated to huFcRn affinity (Giragossian et al. Curr. Drug Metab. 14 (2013) 764-790).

WO 2013/181087 reported multimeric complexes with improved in vivo stability, pharmacokinetics and efficacy.

US 2017/0037121 reported a polypeptide comprising a first polypeptide and a second polypeptide each comprising in N-terminal to C-terminal direction at least a portion of an immunoglobulin hinge region, which comprises one or more cysteine residues, an immunoglobulin CH2-domain and an immunoglobulin CH3-domain, wherein i) the first and the second polypeptide comprise the mutations H310A, H433A and Y436A, or ii) the first and the second polypeptide comprise the mutations L251D, L314D and L432D, or iii) the first and the second polypeptide comprise the mutations L251S, L314S and L432S.

US 2020/0353078 reported isolated IL-33 proteins, active fragments thereof and antibodies, antigen binding fragments thereof, against IL-33 proteins. Also provided are methods of modulating cytokine activity, e.g., for the purpose of treating immune and inflammatory disorders.

Vaughn, D. E., et al. reported the structural basis of pH-dependent antibody binding by the neonatal Fc receptor (Structure 6 (1998) 63-73).

Thus, there is a need for analyzing antibody-FcRn interaction, especially in a technologically more advanced way to address the underlying complexity.

### SUMMARY OF THE INVENTION

While surface plasmon resonance (SPR) is commonly used to measure binding of IgG to recombinant neonatal Fc-receptor (FcRn), it is not straight forward to interpret the data to obtain reliable binding kinetics. Herein is reported a novel SPR based FcRn binding assay for appropriate FcRn binding assessments. This assay can be combined with a suitable visualization, to gain an in-depth understanding of the contribution of the Fc-region and Fab arms of an antibody to FcRn binding kinetics.

That is, herein is reported a novel SPR-based antibody-FcRn binding assay that accounts for the individual Fab-FcRn and Fc-region-FcRn interactions for antibody-FcRn binding assessment. This aspect of the invention is based, at least in part, on the finding of the effect of a pH dependent FcRn coating on a solid phase, such as, e.g., an SPR-chip.

Herein is reported a method for determining antibody-FcRn-interaction, wherein
the FcRn-immobilization surface is an SPR chip, wherein the capture groups are attached directly to the solid surface (layer), and wherein the (solid) surface comprises no dextran matrix/groups (is not derivatized with dextran),
the capture reagent is provided in an isolated, i.e. not dimerized or multimerized, form, i.e. the capture reagent has only a single binding site for the analyte (target molecule), thus the capture reagent is monomeric, and
the running buffer used in the immobilization controls the aggregation state of the capture reagent during immobilization, i.e. if it is maintained in monomeric form or as a dimer/multimer.

One aspect of the current invention is a method for determining antibody-FcRn-interaction comprising the steps of
a) immobilizing FcRn on a solid surface, which is suitable for surface plasmon resonance measurement,
b) individually applying to the solid surface obtained in step a) solutions comprising the antibody at different concentrations and determining the association rate constant as well as the dissociation rate constant for the antibody-FcRn-interaction for each concentration,
c) determining with the rates obtained in step b) the K_{D}-value of the antibody-FcRn-interaction,
   wherein the immobilized FcRn is monomeric FcRn,
   wherein the monomeric FcRn is immobilized (using functional (capture) groups that are) directly (attached) on said solid surface,
   wherein the solid surface is free of branched glucan, and
   wherein the immobilization of the FcRn is effected/done at a pH value of from pH 7 to pH 8.

In one embodiment of the above-mentioned as well as below-mentioned embodiments and aspects, the immobilization of the FcRn is effected/done at a pH value of about pH 7.4.

In one embodiment of the above-mentioned as well as below-mentioned embodiments and aspects, the FcRn is immobilized at a density of 50-150 response units (RU). In one preferred embodiment, the FcRn is immobilized at a density of 80-120 RU.

In one embodiment of the above-mentioned as well as below-mentioned embodiments and aspects, the FcRn is a single chain FcRn (scFcRn). In one embodiment, the scFcRn is a fusion polypeptide of beta-2-microglobulin and human FcRn polypeptide, which are conjugated to each other by a (GGGGS)₄-peptidic linker, and which comprises a C-terminal His(10)-Avi-tag (SEQ ID NO: 07).

In one embodiment of the above-mentioned as well as below-mentioned embodiments and aspects, the FcRn is immobilized using amine coupling at a density of about 1 pg or more, in certain embodiments of about 10 pg or more, in certain embodiments of about 50-150 pg (sc)FcRn per mm 2 chip surface. In one preferred embodiment, the FcRn is immobilized using amine coupling at a density of about pg (sc)FcRn/mm² chip surface.

In one embodiment of the above-mentioned as well as below-mentioned embodiments and aspects, the FcRn is immobilized using biotin/streptavidin coupling at a density of about 1 pg or more, in certain embodiments of about 10 pg or more, in certain embodiments of about 50-150 pg (sc)FcRn per mm 2 chip surface.

In one embodiment of the above-mentioned as well as below-mentioned embodiments and aspects, the immobilization is done with a solution comprising FcRn at a concentration of about 250 μg/ml in 10 mM HEPES buffer at a pH value of about pH 7.4.

In one embodiment of the above-mentioned as well as below-mentioned embodiments and aspects, the solution of the antibody applied to the immobilized FcRn in step b) comprises i) 150 mM NaCl, or ii) 400 mM NaCl, or iii) 400 mM NaCl and 20% (w/w) ethylene glycol.

In one embodiment of the above-mentioned as well as below-mentioned embodiments and aspects, step b) is performed i) with a solution of the antibody comprising 150 mM NaCl, and ii) with a solution of the antibody comprising 400 mM NaCl or/and with a solution of the antibody comprising 400 mM NaCl and 20% (w/w) ethylene glycol. In one embodiment, the solution comprising 400 mM NaCl or/and the solution comprising 400 mM NaCl and 20% (w/w) ethylene glycol is reduces or eliminates Fab-FcRn interactions. In one embodiment, the solution comprising 400 mM NaCl or/and the solution comprising 400 mM NaCl and 20% (w/w) ethylene glycol is used to reduce or eliminate inter-molecular interactions and Fab-FcRn interactions. Thereby determination of the isolated Fc-region-FcRn-interaction is achieved.

In one embodiment of the above-mentioned as well as below-mentioned embodiments and aspects, the solution of the antibody applied to the immobilized FcRn in step b) comprises either 10 mM MES, 150 or 400 mM NaCl, 0.05% (w/v) polysorbate 20 (P-20) and optionally 20% (w/w) ethylene glycol at pH value of pH 5.8, or comprises 10 mM HEPES, 150 mM or 400 mM NaCl, 0.05% (w/v) P-20 and optionally 20% (w/w) ethylene glycol at a pH value of pH 7.4.

In one embodiment of the above-mentioned as well as below-mentioned embodiments and aspects, the branched glucan is a complex branched glucan. A glucan is a polysaccharide obtained by the condensation of glucose. In one embodiment, the complex branched glucan is dextran. In one embodiment, the complex branched glucan is a branched poly-α-d-glucoside of microbial origin having glycosidic bonds predominantly from C-1 to C-6″. In one embodiment, the dextran has a molecular weight of from 3 kDa to 2,000 kDa.

In one embodiment of the above-mentioned as well as below-mentioned embodiments and aspects, the Fab-FcRn interaction as well as the Fc-region-FcRn interaction are divided and visualized using a 2-/3-dimensional diagram, wherein the stability (log kd, off-rate) is shown/corresponds to the x-axis and the recognition (log ka, on-rate) is shown/corresponds to the y-axis.

In one embodiment of the above-mentioned as well as below-mentioned embodiments and aspects, the interaction between FcRn and the Fc-region of an antibody is to be analyzed,
the sensor surface is an SPR chip with carboxymethylated surface, wherein the carboxyl groups are attached directly to the (solid) surface (layer), and wherein the SPR chip is free of a dextran matrix,
a beta-2-microglobulin-human FcRn fusion polypeptide (the groups are linked by a (GGGGS)₄-peptidic linker) comprising a C-terminal His(10)-Avi-tag is immobilized on the sensor surface using amine coupling at neutral pH (about 250 μg/ml in 10 mM HEPES at pH 7.4), and
the running buffer is either 10 mM MES, 150 mM NaCl, pH 5.8, 0.05% (w/v) P-20 or 10 mM HEPES, pH 7.4, 150 mM NaCl, 0.05% (w/v) P-20.

In one embodiment of the above-mentioned as well as below-mentioned embodiments and aspects, the sensor surface is an SPR chip with carboxymethylated surface, wherein the carboxyl groups are attached directly to the (solid) surface (layer) and which is free of dextran. In this embodiment, a beta-2-microglobulin-human FcRn fusion polypeptide (the groups are linked by a (GGGGS)₄-peptidic linker) comprising a C-terminal His(10)-Avi-tag is immobilized to the (solid) surface using amine coupling at neutral pH (about 250 μg/ml in 10 mM HEPES at pH 7.4) and the employed running buffer is either 10 mM MES, 150 mM NaCl, pH 5.8, 0.05% (w/v) P-20 or 10 mM HEPES, pH 7.4, 150 mM NaCl, 0.05% (w/v) P-20).

In one embodiment of the above-mentioned as well as below-mentioned embodiments and aspects, the method is for selecting an antibody with pH-dependent FcRn-mediated antibody recycling or/and long in vivo half-life and the antibody that is selected has a pH-dependent overall antibody-FcRn interaction strength in the range of 100-400 nM at pH 5.5-6.0. The overall antibody-FcRn interaction strength comprises the Fc-FcRn- and the Fab-FcRn interaction.

In one embodiment of the above-mentioned as well as below-mentioned embodiments and aspects, the method is for selecting an antibody with pH-dependent FcRn-mediated antibody recycling or/and long in vivo half-life and the antibody that is selected has a one sided Fc-region-FcRn binding strength of 25 nM or higher at a pH value in the range from pH 5.5 to pH 6.5. In one embodiment, the binding strength is 100 nM or higher, or 200 nM or higher, or 300 nM or higher. In one embodiment, a binding strength lower than 100 (200) nM for one sided Fc-region-FcRn binding affinity is used if no additional Fab-FcRn binding affinity is present, especially at pH 7.4. This should be connected to a low or non-detectable binding strength at pH 7.4 and higher. In one embodiment, the binding strength is 25 nM or higher and the antibody dissociates from FcRn at pH 7.4. This can be used for the selection of antibody variants with improved pharmacokinetic properties.

In one embodiment of the above-mentioned as well as below-mentioned embodiments and aspects, the method is for selecting a variant antibody with modified Fc-region, wherein the method comprises conducting/performing step b) with the parent antibody and at least two variant antibodies differing in their Fc-region amino acid sequence, step c) is determining the interaction spot pattern, and a variant antibody is selected that has an interaction spot pattern that is similar/matches the interaction spot pattern of the parent antibody, wherein the spot pattern is a 2-/3-dimensional diagram, in which the stability (log kd, off-rate) is shown/corresponds to the x-axis and the recognition (log ka, on-rate) is shown/corresponds to the y-axis.

In one embodiment of the above-mentioned as well as below-mentioned embodiments and aspects, the method is for determining the kind of Fab-FcRn-interaction, i.e. for determining whether a charge- or hydrophobicity-based interaction is present, wherein the method comprises conducting/performing step b) first with the antibody in a solution comprising 10 mM MES or HEPES, 150 mM, % (w/v) P-20 at a pH value of pH 7.4 to obtain a first interaction spot pattern, second with the antibody in a solution comprising 10 mM MES or HEPES, 400 mM, % (w/v) P-20 at a pH value of pH 7.4 to obtain a second interaction spot pattern, and third with the antibody in a solution comprising 10 mM MES or HEPES, 400 mM, 0.05% (w/v) P-20 and 20% (w/w) ethylene glycol at a pH value of pH 7.4 to obtain a third interaction spot pattern, wherein the kind of Fab-FcRn-interaction is charge-based if the first interaction spot pattern and the second interaction spot pattern are different, and the kind of Fab-FcRn-interaction is hydrophobicity-based if the first and the second interaction spot pattern are similar and the third interaction spot pattern is different, wherein the interaction spot pattern is a 2-/3-dimensional diagram, in which the stability (log kd, off-rate) is shown/corresponds to the x-axis and the recognition (log ka, on-rate) is shown/corresponds to the y-axis.

In one embodiment of the above-mentioned as well as below-mentioned embodiments and aspects, the antibody is a bispecific antibody.

In one embodiment of the above-mentioned as well as below-mentioned embodiments and aspects, the bispecific antibody is a domain exchanged antibody.

In one embodiment of the above-mentioned as well as below-mentioned embodiments and aspects, the bispecific antibody is a one-armed single chain antibody.

In one embodiment of the above-mentioned as well as below-mentioned embodiments and aspects, the bispecific antibody is a two-armed single chain antibody.

In one embodiment of the above-mentioned as well as below-mentioned embodiments and aspects, the bispecific antibody is a common light chain bispecific antibody.

One aspect of the current invention is the method according to the current invention for selecting an antibody/antibody selection. In one embodiment, the method according to the current invention is performed with at least two antibodies differing in their FcRn interaction, whereby the antibody is selected that has the highest/the antibodies are selected that have higher affinity/strength of the (isolated) Fc-region-FcRn-interaction.

One aspect of the current invention is the method according to the current invention for antibody engineering. In one embodiment, the method according to the invention is performed with at least two antibodies differing in their Fc-region- and/or Fab-amino acid sequence, whereby the antibody is selected that has the largest/biggest ratio/antibodies are selected that have largest/bigger ratio between Fc-FcRn-interaction and Fab-FcRn-interaction.

One aspect of the current invention is the use of the method according to the invention for the determination of the Fab-FcRn and Fc-region-FcRn interaction.

One aspect of the current invention is the use of the method according to the invention for determining the effect of an antibody-Fc-region mutation on in vivo half-life of the antibody.

One aspect of the current invention is the use of the method according to the invention for selecting an antibody with modified/improved (longer or shorter) in vivo half-life.

One aspect of the current invention is the use of the method according to the invention for determining Fab-FcRn-interaction and Fc-region-FcRn-interaction of an antibody.

One aspect of the current invention is the use of the method according to the invention for delineating Fab-FcRn-interaction and Fc-region-FcRn-interaction of an antibody.

One aspect of the current invention is the use of the method according to the invention for separately analyzing Fab-FcRn-interaction and Fc-region-FcRn-interaction of an antibody.

It is herewith expressly pointed out that the combination of any aspect with any individual embodiment or a combination of embodiments even if not present verbatim is also disclosed. An aspect as reported herein relates to an individual, independent way of performing the current invention, whereas an embodiment relates to a specific, dependent way of performing one or more aspects of the current invention.

### DETAILED DESCRIPTION OF THE INVENTION

Herein is reported a novel SPR-based antibody-FcRn binding assay that accounts for individual Fab-FcRn and Fc-region-FcRn interactions in antibody-FcRn binding assessment. This aspect of the invention is based, at least in part, on the pH dependent FcRn coating on a solid phase, such as, e.g., an SPR-chip.

The method according to this aspect of the invention has been confirmed by reducing the complexity of the antibody down to the Fc-region alone with just one active FcRn binding site and thereafter adding back the additional domains of the molecule one after another.

The current invention is based, at least in part, on the finding that the SPR setup for determining Fc-region-FcRn interaction comprises many variabilities.

The current inventions is further based, at least in part, on the finding that by using a deliberate immobilization of FcRn on the SPR sensor surface, i.e. by using FcRn capture, in combination with a deliberate buffer setup, information about all antibody-FcRn interactions, i.e. of Fab-FcRn and Fc-region-FcRn interactions, can be obtained.

The invention is based, at least in part, on the finding that the manifold IgG-FcRn interaction have to be interpreted/seen in concert. Only after dissection into the individual binding steps and binding interactions it is possible to understand the individual molecular interactions that contribute to the overall binding. Only based on this dissection of the interactions an antibody can successfully be engineered, i.e. by engineering the Fc-FcRn and Fab-FcRn interaction separately.

The invention is based, at least in part, on the finding that, due to the symmetry of the antibody heavy chains, a mixture of different binding events take place and it is important to immobilize a controlled, i.e. defined, amount of FcRn on the SPR sensor surface. This is achieved in the method according to the invention by controlling FcRn dimerization, e.g. formation of a heterodimer, during the immobilization step. It has been found that FcRn dimerizes in a pH-dependent manner. By using a single chain FcRn a very homogeneous surface of FcRn on the SPR sensor surface can be provided.

The current invention is based, at least in part, on the finding that i) by controlling the SPR chip, especially the use of a single chain FcRn and immobilization at neutral/physiological pH (i.e. in the range of pH 7 to pH 8), and ii) adjusting the buffer conditions, the multistage binding mechanism between an antibody and FcRn can be dissected and used for the selection and screening of engineered antibodies with respect to pharmacokinetic properties. In certain embodiments, the antibody is simplified, and/or an adequate visualization with the stability (log kd) on the x-axis and the recognition (log ka) on the y-axis is used to separate the different antibody-FcRn interactions, and/or the pharmacokinetic property is pH-dependent FcRn-binding.

The current invention is based, at least in part, on the finding that the pH-dependent antibody-FcRn interaction strength has to be in the range of 100-400 nM at pH 5.5-6.0 for selecting an antibody with suitable pH-dependent FcRn-mediated antibody recycling and thereby long in vivo half-life. In certain embodiments, the antibody-FcRn interaction strength is the overall antibody-FcRn interaction strength. The overall antibody-FcRn interaction strength comprises the Fc-FcRn- and the Fab-FcRn interaction. In certain embodiments, the antibody-FcRn interaction strength is the Fc-FcRn interaction strength.

The current invention is based, at least in part, on the finding found that the fraction of the population with higher affinity, i.e. the spot closer to the origin (lower left corner of the 2-/3-dimensional diagram), increases with higher density of immobilized scFcRn (single-chain FcRn) on the SPR solid surface, i.e. chip.

### Definitions

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991). The amino acid positions of all constant regions and domains of the heavy and light chain can be numbered according to the Kabat numbering system described in Kabat, et al., Sequences of Proteins of Immunological Interest, ed., Public Health Service, National Institutes of Health, Bethesda, MD (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein.

It is also to be noted that the terms “comprising”, “including”, and “having” can be used interchangeably.

To a person skilled in the art procedures and methods are well known to convert an amino acid sequence, e.g. of a peptidic linker or fusion polypeptide, into a corresponding encoding nucleic acid sequence. Therefore, a nucleic acid is characterized by its nucleic acid sequence consisting of individual nucleotides and likewise by the amino acid sequence of a peptidic linker or fusion polypeptide encoded thereby.

The use of recombinant DNA technology enables the generation derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The term “about” denotes a range of +/−20% of the thereafter following numerical value. In one embodiment the term about denotes a range of +/−10% of the thereafter following numerical value. In one embodiment the term about denotes a range of +/−% of the thereafter following numerical value.

“Affinity” or “binding affinity” refers to the strength of the sum total of non-covalent interactions between a single binding site of a molecule (e.g. an antibody) and its binding partner (e.g. an antigen). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

The term “antibody” herein is used in the broadest sense and encompasses various antibody structures, including but not limited to monoclonal antibodies, multispecific antibodies (e.g. bispecific antibodies, trispecific antibodies), and antibody fragments so long as they comprise at least an Fc-region.

An antibody in general comprises two so called light chain polypeptides (light chain) and two so called heavy chain polypeptides (heavy chain). Each of the heavy and light chain polypeptides contains a variable domain (variable region) (generally the amino terminal portion of the polypeptide chain) comprising binding regions that are able to interact with an antigen. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (C1q). The constant domains of an antibody heavy chain comprise the CH1-domain, the CH2-domain and the CH3-domain, whereas the light chain comprises only one constant domain, CL, which can be of the kappa isotype or the lambda isotype.

The variable domain of an immunoglobulin's light or heavy chain in turn comprises different segments, i.e. four framework regions (FR) and three hypervariable regions (HVR).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “binding (to FcRn)” denotes the binding of an antibody or at least an antibody Fc-region or an antibody Fc-region comprising fusion polypeptide to the (human) FcRn in an in vitro assay. In one embodiment, binding is determined in a binding assay in which the (human) FcRn is bound to a solid surface, e.g. a sensor chip, and binding of the antibody (or isolated Fc-region or Fc-region comprising fusion polypeptide) is measured by Surface Plasmon Resonance (SPR).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al.

The term “valent” as used within the current application denotes the presence of a specified number of binding sites in a (antibody) molecule. As such, the terms “bivalent”, “tetravalent”, and “hexavalent” denote the presence of two binding site, four binding sites, and six binding sites, respectively, in a (antibody) molecule. The bispecific antibodies as reported herein are in one preferred embodiment “bivalent”.

The term “binding affinity” denotes the strength of the interaction of a single binding site with its respective target. Experimentally, the affinity can be determined e.g. by measuring the kinetic constants/rates for association (kA) and dissociation (kd) of the antibody and FcRn in the equilibrium.

The term “binding avidity” denotes the combined strength of the interaction of multiple binding sites of one molecule (antibody) with the same target. As such, avidity is the combined synergistic strength of bond affinities rather than the sum of bonds. Requisites for avidity are: polyvalency of a molecule, such as an antibody, or a functional multimer of one target (FcRn).

The complex (mono or bivalent) Fc-association does not differ between affine and avid binding. However, the complex dissociation for avid binding depends on the simultaneous dissociation of all binding sites involved. Therefore, the increase of binding strength due to avid binding (compared to affine binding) depends on the dissociation kinetics/complex stability: the bigger (higher) the complex stability, the less likely is the simultaneous dissociation of all involved binding sites; for very stable complexes, the difference of affine vs. avid binding becomes essentially zero;—the smaller (lower) the complex stability, the more likely is the simultaneous dissociation of all involved binding sites; the difference of affine vs. avid binding is increased.

Multispecific Antibodies

In certain embodiments, the antibody used in the method according to the invention is a multispecific antibody, e.g. a bispecific antibody. Multispecific antibodies are monoclonal antibodies that have binding specificities for at least two different sites on one antigen or for at least two different antigens. In certain embodiments, one of the binding specificities is for a first antigen and the other is for a different second antigen. In certain embodiments, multispecific antibodies may bind to two different epitopes of the same antigen.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004).

The antibody can also be a multispecific antibody as described in WO 2009/080251, WO 2009/080252, WO 2009/080253, WO 2009/080254, WO 2010/112193, WO 2010/115589, WO 2010/136172, WO 2010/145792, or WO 2010/145793.

The antibody thereof may also be a multispecific antibody as disclosed in WO 2012/163520 (also referred to as “DutaFab”).

Bispecific antibodies are generally antibody molecules that specifically bind to two different, non-overlapping epitopes on the same antigen or to two epitopes on different antigens.

Different bispecific antibody formats are known.

Exemplary bispecific antibody formats which can be used in the methods as reported herein are
the domain-exchanged antibody (CrossMab format): a multispecific IgG antibody comprising a first Fab fragment and a second Fab fragment, wherein in the first Fab fragment
   a) only the CH1 and CL domains are replaced by each other (i.e. the light chain of the first Fab fragment comprises a VL and a CH1 domain and the heavy chain of the first Fab fragment comprises a VH and a CL domain);
   b) only the VH and VL domains are replaced by each other (i.e. the light chain of the first Fab fragment comprises a VH and a CL domain and the heavy chain of the first Fab fragment comprises a VL and a CH1 domain); or
   c) the CH1 and CL domains are replaced by each other and the VH and VL domains are replaced by each other (i.e. the light chain of the first Fab fragment comprises a VH and a CH1 domain and the heavy chain of the first Fab fragment comprises a VL and a CL domain); and
wherein the second Fab fragment comprises a light chain comprising a VL and a CL domain, and a heavy chain comprising a VH and a CH1 domain;
the domain exchanged antibody may comprises a first heavy chain including a CH3 domain and a second heavy chain including a CH3 domain, wherein both CH3 domains are engineered in a complementary manner by respective amino acid substitutions, in order to support heterodimerization of the first heavy chain and the modified second heavy chain, e.g. as disclosed in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, or WO 2013/096291 (incorporated herein by reference);
a one-armed single chain antibody (one-armed single chain format): antibody comprising a first binding site that specifically binds to a first epitope or antigen and a second binding site that specifically binds to a second epitope or antigen, whereby the individual chains are as follows
   light chain (variable light chain domain+light chain kappa constant domain)
   combined light/heavy chain (variable light chain domain+light chain constant domain+peptidic linker+variable heavy chain domain+CH1+Hinge+CH2+CH3 with knob mutation)
   heavy chain (variable heavy chain domain+CH1+Hinge+CH2+CH3 with hole mutation);
two-armed single chain antibody (two-armed single chain format): antibody comprising a first binding site that specifically binds to a first epitope or antigen and a second binding site that specifically binds to a second epitope or antigen, whereby the individual chains are as follows
   combined light/heavy chain 1 (variable light chain domain+light chain constant domain+peptidic linker+variable heavy chain domain+CH1+Hinge+CH2+CH3 with hole mutation)
   combined light/heavy chain 2 (variable light chain domain+light chain constant domain+peptidic linker+variable heavy chain domain+CH1+Hinge+CH2+CH3 with knob mutation);
a common light chain bispecific antibody (common light chain bispecific antibody format): antibody comprising a first binding site that specifically binds to a first epitope or antigen and a second binding site that specifically binds to a second epitope or antigen, whereby the individual chains are as follows
   light chain (variable light chain domain+light chain constant domain)
   heavy chain 1 (variable heavy chain domain+CH1+Hinge+CH2+CH3 with hole mutation)
   heavy chain 2 (variable heavy chain domain+CH1+Hinge+CH2+CH3 with knob mutation).

In one embodiment the bispecific antibody is a domain exchanged antibody.

In one embodiment the bispecific antibody is a one-armed single chain antibody.

In one embodiment the bispecific antibody is a two-armed single chain antibody.

In one embodiment the bispecific antibody is a common light chain bispecific antibody.

Surface-Plasmon-Resonance Methods

Kinetic binding parameters of antibodies to FcRn can be investigated by surface plasmon resonance, e.g. using a BIAcore instrument (GE Healthcare Biosciences AB, Uppsala, Sweden).

Generally, for affinity measurements of antibodies to their target antigen an anti-IgG antibody, e.g. an anti-human IgG or an anti-mouse IgG antibody, is immobilized on a sensor chip, such as a CM5 chip, via amine coupling for capture and presentation of the respective antibody to be analyzed.

For example, about 2,000-12,000 response units (RU) of a 10-30 μg/ml anti-IgG antibody is coupled onto some spots of the flow cells (e.g. spots 1 and 5 are active and spots 2 and 4 are reference spots, or spots 1 and 2 are reactive and spots 3 and 4 are reference spots, etc.) of a CM5 sensor chip in a BIAcore B4000 or T200 instrument at pH 5.0 at 10-30 μl/min, by using an amine coupling kit supplied by GE Healthcare.

Binding of the antibody to its cognate antigen can be determined in HBS buffer (HBS-P (10 mM HEPES, 150 mM NaCl, 0.005% Tween 20, pH 7.4), or HBS-EP+buffer (0.01 M HEPES, 0.15 M NaCl, 3 mM EDTA, 0.05% v/v Surfactant PS20, pH 7.4), or HBS-ET buffer (10 mM HEPES pH 7.4, 150 mM NaCl, 3 mM EDTA, % w/v Tween 20)), at 25° C. (or alternatively at a different temperature in the range from 12° C. to 37° C.).

Thus, the antibody is injected in the respective buffer for 30 seconds with a concentration in the range of 10 nM to 1 μM and binds to the reactive spots of each flow cell.

Thereafter the corresponding antigen is injected in various concentrations in solution, such as e.g. 144 nM, 48 nM, 16 nM, 5.33 nM, 1.78 nM, 0.59 nM, 0.20 nM and 0 nM, depending on the affinity of the antibody and association is determined by with injection times of 20 seconds to 10 minutes at 10-30 μl/min. flow rate.

Dissociation is determined by washing the chip surface with the respective buffer for 3-10 minutes.

A K_{D} value is estimated using a 1:1 Langmuir binding model using the manufacturer's software and instructions. Negative control data (e.g. buffer curves) are subtracted from sample curves for correction of system intrinsic baseline drift and for noise signal reduction.

### Specific Embodiments of the Invention

Due to the antibody architecture, the FcRn binding on a molecular level is very complex.

Thus, antibodies with the same IgG1 Fc-region but different Fabs show a different behavior in their FcRn interaction (see FIG. 1; SPR sensorgram of different humanized/chimeric human IgG1 Fc-region comprising antibodies (exploratory and approved); sensorgrams were recorded under the same SPR conditions with the same concentration and same monomer concentration; the only difference is the antigen binding site).

The invention is based, at least in part, on the finding that the FcRn setup comprises many variabilities and that immobilization of FcRn on the SPR sensor surface, i.e. by using FcRn capture, information about all antibody-FcRn interactions, i.e. of Fab and Fc-region, can be obtained.

The invention is based, at least in part, on the finding that it is detrimental not to interpret the manifold IgG-FcRn interaction in concert, by classical KD interpretation. Only after dissection into the individual binding steps it is possible to understand the molecular interactions that contribute to the binding. Only out of this understanding the needed engineering can be applied in the meaning of adaptive Fab of Fc engineering.

The invention is based, at least in part, on the finding that due to the symmetry of the antibody heavy chains there is a mixture of different binding events and it is therefore important to immobilize a controlled, i.e. defined, amount of FcRn on the SPR sensor surface. This is achieved by controlling the FcRn dimerization, e.g. formation of a heterodimer. It has been found that FcRn dimerizes in a pH-dependent manner. By using a single chain FcRn a very homogeneous surface of FcRn on the SPR sensor surface can be provided.

The antibody-FcRn interactions, i.e. the Fab-FcRn interaction as well as the Fc-Region-FcRn interaction, can be divided and visualized using a 2-/3-dimensional diagram, wherein the stability (log kd, off-rate) corresponds to the x-axis and the recognition (log ka, on-rate) corresponds to the y-axis (see, e.g., FIG. 5). For the generation of such a 2-/3-dimensional diagram any suitable software can be used, such as, e.g., the Interaction Map (IM) software of Ridgeview Diagnostics AB (Uppsala, Sweden).

Thus, coming from theory, there should be only one spot if the interaction between an Fc-region that has exactly one binding site with/for FcRn is analyzed.

In FIG. 2, a 3-dimensional diagram (with the stability (log kd) on the x-axis, the recognition (log ka) on the y-axis and intensity on the z-axis) of an isolated, Fab-less Fc-region-FcRn-interaction, i.e. a theoretical 1:1 interaction of an isolated antibody Fc-region with a single FcRn-binding site obtained by introducing the mutations 1253A/H310A/H435A (numbering according to Kabat is used herein) in one Fc-region polypeptide, is shown. It can be seen that, despite the underlying theory, two spots are present, although one Fc-region polypeptide is inert with respect to FcRn-interaction, i.e. it cannot bind to FcRn. It has been found that the portion of the population with higher affinity, i.e. the spot closer to the origin (lower left corner of the diagram), increases with higher density of immobilized scFcRn (single-chain FcRn) on the SPR solid surface, i.e. chip.

In FIG. 3, a 2-dimensional diagram of the interaction of a full-length, monospecific anti-digoxygenin antibody with FcRn on an SPR solid surface is shown. It can be seen that in this case even three spots are visible.

The difference of the interaction as shown in the two examples beforehand, is, at least in part, due to the interaction mode, i.e., if it is a “complex” or “simple” interaction, respectively, of the antibody with FcRn.

Thus, in order to resolve all these interactions properly, an improved SPR method has to be employed.

Such an improved method is provided in the current invention.

In more detail, the current invention provides a method for detecting antibody-FcRn-interaction, wherein
the immobilization surface is an SPR chip with a surface, wherein the capture groups are attached directly to the surface layer and which has no dextran matrix,
the capture reagent is provided in an isolated, i.e. not dimerized or mutimerized, form, i.e. the capture reagent has only a single binding site for the analyte (target molecule),
the running buffer used in the immobilization controls the aggregation state of the capture reagent during immobilization, i.e. if it is maintained in monomeric form or as a dimer/multimer.

With this method a low amount of capture reagent, i.e. in the range of 50-150 RU, can be covalently conjugated to the solid surface

Thus, in one aspect of the current invention, wherein the interaction between FcRn and an Fc-region/antibody is to be analyzed,
the sensor surface is an SPR chip with carboxymethylated surface, wherein the carboxyl groups are attached directly to the surface layer and which has no dextran matrix,
a beta-2-microglobulin-human FcRn fusion polypeptide (the groups are linked by a (GGGGS)₄-peptidic linker) comprising a C-terminal His(10)-Avi-tag is immobilized on the sensor surface using amine coupling at neutral pH (about 250 μg/ml in 10 mM HEPES at pH 7.4), and
the running buffer is either 10 mM MES, 150 mM NaCl, pH 5.8, 0.05% P-20 or HBS-P buffer (10 mM HEPES buffer, pH 7.4, 150 mM NaCl, 0.05% P-20).

With the method according to the invention a low amount of scFcRn, i.e. about 80-120 RU or 50-100 RU, can be covalently conjugated to the solid surface. Since immobilization is carried out at pH 7.4, it is ensured that that the scFcRn is immobilized in monomeric form and does not form aggregates. The effect thereof is shown in FIG. 4, wherein the sensorgram and in FIG. 5, wherein the corresponding 2-dimensional diagram (with the stability (log kd) on the x-axis and the recognition (log ka) on the y-axis) of a simple Fc-region-FcRn-interaction, i.e. a 1:1 interaction of an isolated, Fab-less antibody Fc-region with a single FcRn-binding site obtained by introducing the mutations I253A/H310A/H435A in one Fc-region polypeptide and maintaining the corresponding wild-type Fc-region polypeptide as the respective other Fc-region polypeptide at low FcRn immobilization levels with amine coupling are shown. It can be seen that only a single spot is present.

In contrast thereto, if the immobilization of the scFcRn is performed at pH 5.5 then dimeric scFcRn is immobilized due to heterodimerization occurring at this pH value. Compared to the interaction with monomeric scFcRn, signal intensities are twice as high showing that dimeric scFcRn can bind two Fc-regions.

In FIG. 6 the sensorgram of a complex IgG1 full length antibody-FcRn-interaction is shown. In FIG. 7 and FIG. 8, the 2-dimensional diagrams of a complex IgG1 full length antibody-FcRn-interaction at low FcRn immobilization levels using amine coupling as well as at high FcRn immobilization levels using biotin/avidin coupling, respectively, are shown. It can be seen that additional spots and thereby interactions beside the Fc-region-FcRn interaction are present.

Thus, by adding the Fabs to the Fc-region (mutations I253A/H310A/H435A in one Fc-region polypeptide and wild-type in the respective other Fc-region polypeptide) the effect of isolated Fab-FcRn-interaction can be determined.

In a first example, in FIG. 9 to FIG. 11, this is shown with anti-digoxygenin-Fabs added to the Fc-region with a single FcRn binding site (mutations I253A/H310A/H435A in one Fc-region polypeptide and wild-type in the respective other Fc-region polypeptide; for a sketch of the antibody see FIG. 42-a). Depending on the employed buffer conditions, the interaction can be strengthened or weakened (low density FcRn, about 80 RU):
150 mM sodium chloride (FIG. 9): intramolecular interactions (1250 nM; see FIG. 16, spot 1, for sketch) and intermolecular interactions (20.5 nM; see FIG. 16, spot 2, for sketch);
400 mM sodium chloride (FIG. 10): intramolecular interactions (1060 nM; is strengthened compared to 150 mM sodium chloride) and intermolecular interactions (60 nM);
400 mM sodium chloride and 20% (w/w) ethylene glycol (MW=62.07 g/mol) (FIG. 11): only intramolecular interactions (3230 mM; is weakened compared to the other conditions) and no intermolecular interactions.

Thus, by applying the method according to the invention in combination with high ionic buffer strength the Fab-FcRn can be reduced or even eliminated. Without being bound by this theory, it is assumed that all inter-molecular interactions as well as the Fab-FcRn-interactions are eliminated and the interaction determined under these conditions is the Fc-region-FcRn-interaction.

In a second example, the same change of the interaction strength has also been shown for an asymmetric antibody with the mutations I253A/H310A/H435A in one Fc-region polypeptide (eliminating Fc-FcRn-interaction) and the mutations M252Y/S254T/T256E in the respective other Fc-region polypeptide (increasing the Fc-FcRn-interaction strength) (see FIG. 42-b for sketch of the antibody):
150 mM sodium chloride: intramolecular interactions (184 nM; see FIG. 16, spot 1, for sketch) and intermolecular interactions (4.7 nM; see FIG. 16, spot 2, for sketch);
400 mM sodium chloride: intramolecular interactions (166 nM; is strengthened compared to 150 mM sodium chloride) and intermolecular interactions (6.2 nM).

The same change of the interaction strength has also been determined for an asymmetric antibody with the mutations I253A/H310A/H435A in one Fc-region polypeptide and the mutations T307H/N434H in the respective other Fc-region polypeptide (see FIG. 42-c for sketch of the antibody):
150 mM sodium chloride: intramolecular interactions (391 nM; see FIG. 16, spot 1, for sketch) and intermolecular interactions (10.4 nM; see FIG. 16, spot 2, for sketch);
400 mM sodium chloride: intramolecular interactions (325 nM; is strengthened compared to 150 mM sodium chloride) and intermolecular interactions (4.3 nM and 39 nM).

Thus, increased salt concentrations in the used buffer strengthen the intramolecular interactions in an Fc-region asymmetric antibody. Therefore, in one embodiment, when the determination is of intramolecular interactions of an asymmetric antibody, i.e. a full-length, Y-shaped, bivalent, bispecific antibody, the buffer comprises about 400 mM salt, preferably sodium chloride.

When using the same anti-digoxygenin Fabs connected to a wild-type IgG1 Fc-region then a different effect can be seen (low density FcRn, about 80 RU; FIG. 12 to FIG. 14):
150 mM sodium chloride (FIG. 12): intramolecular binding 1+2 (see FIG. 15 for sketch) at 382 nM, intramolecular binding 1+2+1+2 (see FIG. 15 for sketch) at 0.16 nM; intermolecular binding 1+2′ (see FIG. 15 for sketch) at 5.7 nM, intermolecular binding 1+1′ (see FIG. 15 for sketch) at 126 nM;
400 mM sodium chloride (FIG. 13): intramolecular binding 1+2 (see FIG. 15 for sketch) at 840 nM, intramolecular binding 1+2+1+2 (see FIG. 15 for sketch) at 0.33 nM; intermolecular binding 1+2′ (see FIG. 15 for sketch) at 4.8 nM, intermolecular binding 1+1′ (see FIG. 15 for sketch) at 100 nM; the intramolecular interaction is weakened and the intermolecular interaction is strengthened compared to 150 mM sodium chloride;
400 mM sodium chloride and 20% (w/w) ethylene glycol (MW=62.07 g/mol) (FIG. 14): intramolecular binding 1+2 (see FIG. 15 for sketch) at 1140 nM and intermolecular binding 1+1′ (see FIG. 15 for sketch) at 75 nM; the intramolecular interaction is weakened and the intermolecular interaction is strengthened (i.e. more dominant compared to the other spots) compared to 150 mM sodium chloride.

When using the same anti-digoxygenin Fabs connected to an IgG1 Fc-region with the mutations M252Y/S254T/T256E in both Fc-region polypeptides the same effect can be seen (low density FcRn, about 80 RU) (see FIG. 42-d for sketch of the antibody):
150 mM sodium chloride: intramolecular binding 1+2 (see FIG. 15 for sketch) at 92 nM, intramolecular binding 1+2+1+2 (see FIG. 15 for sketch);
400 mM sodium chloride: intramolecular binding 1+2 (see FIG. 15 for sketch) at 92 nM, intramolecular binding 1+2+1+2 (see FIG. 15 for sketch) at 1.6 nM.

As seen in the increased Fc binding strength the balance between the Fc-FcRn interaction driven spot and the Fab-Fc mediated avidity spot shifts to only Fc-FcRn interaction if the Fc-region is engineered for very strong FcRn-binding.

When using the same anti-digoxygenin Fabs connected to an IgG1 Fc-region with the mutations T307H/N434H in both Fc-region polypeptides the same effect can be seen (low density FcRn, about 80 RU) (for sketch of the antibody see FIG. 42-e):
150 mM sodium chloride: intramolecular binding 1+2 (see FIG. 15 for sketch) at 177 nM, intramolecular binding 1+2+1+2 (see FIG. 15 for sketch) at 0.12 nM; intermolecular binding 1+2′ (see FIG. 15 for sketch) at 3 nM, intermolecular binding 1+1′ (see FIG. 15 for sketch) at 71 nM;
400 mM sodium chloride: intramolecular binding 1+2 (see FIG. 15 for sketch) at 156 nM, intramolecular binding 1+2+1+2 (see FIG. 15 for sketch) at 0.13 nM; intermolecular binding 1+1′ (see FIG. 15 for sketch) at 25 nM.

It has been found that a dramatic increase in antibody-FcRn-interaction by engineering an antibody for enhanced pH-dependent FcRn interaction not inevitably results in likewise improved pharmacokinetic properties.

| Fc-region | CL [mL/d/kg] | T½ [d] |
|---|---|---|
| IgG1 wt | 3.82 | 15.6 |
| symmetric T307H/N434H | 3.24 | 18.1 |
| symmetric M252Y/S254T/T256E | 3.45 | 23.0 |

Additionally, it has been found that having an interaction spot pattern that matches the parental antibody is preferred over a shifted pattern for pharmacokinetic engineering of an antibody, e.g. when introducing the YTE mutations as the antibody might have a modified thermostability (see FIG. 36).

The complex, multistep antibody-FcRn binding mechanism is a multivariate mechanism involving
pH dependent affinity: FcRn binding cannot be described by a simple 1:1 interaction;
pH dependent avidity: both Fc-region heavy chains are involved in the Fc-FcRn interaction;
Fab contribution: due to additional and simultaneously Fab-FcRn interaction several interactions sum up to a heterogeneous binding pattern.

Thus, a two-pronged binding mechanism of IgG to the neonatal Fc-receptor controls complex stability and IgG serum half-life. The complexity is visualized in FIG. 15 and FIG. 16.

The current invention is based, at least in part, on the finding that i) by controlling the SPR chip, especially the use of a single chain FcRn and immobilization at neutral/physiological pH (i.e. in the range of pH 7 to pH 8), and ii) adjusting the buffer conditions, the multistage binding mechanism between an antibody and FcRn can be used for the selection and screening of engineered antibodies with respect to a pharmacokinetic property. In certain embodiments, the antibody is simplified, and/or an adequate visualization with the stability (log kd) on the x-axis and the recognition (log ka) on the y-axis is used to separate the different antibody-FcRn interactions, and/or the pharmacokinetic property is pH-dependent FcRn-binding.

Thus, the measurements conducted with the method according to the current invention are done with monomeric immobilized FcRn. Thereby it is possible to resolve
the influence of binding/interaction valency
the influence of Fab-charges
the influence of FcRn-density on the chip surface
the influence of buffer composition.

First, by using amine coupling or biotin/avidin coupling for immobilizing the (sc)FcRn to the surface of the SPR chip the coating density is controlled down to low levels (see FIG. 19 to FIG. 26), i.e. the FcRn density on the chip surface is reduced compared to other methods. Thereby the sensitivity of the method is increased and different interactions can be visualized in an individualized form. By using a 2- or 3-dimensional diagram with the stability (log kd) on the x-axis and the recognition (log ka) on the y-axis, i.e. by an adequate visualization, the influence of, on the one hand, Fc-engineering and, on the other hand, of the Fab-FcRn interaction on the overall antibody-FcRn interaction can be visualized. Especially the interrelation of Fc- and Fab-FcRn binding strength can be separated (see FIG. 17 and FIG. 18).

For the generation of such a 2- or 3-dimensional diagram any suitable software can be used, such as, e.g., the Interaction Map (IM) software of Ridgeview Diagnostics AB (Uppsala, Sweden).

In FIG. 21 and FIG. 22 the resolution obtained with biotin/avidin coupling at a coating density of about 1700 RU is shown. In FIG. 23 and FIG. 24 the resolution obtained with biotin/avidin coupling at a coating density of about 80 RU is shown. In FIG. 25 and FIG. 26 the resolution obtained with amine coupling at a coating density of about 80 RU is shown.

In one preferred embodiment, the coating density using amine coupling in the method according to the current invention is about 80-115 pg (sc)FcRn/mm² chip surface (corresponding to 80-115 RU).

In one preferred embodiment, the coating density using biotin/avidin coupling in the method according to the current invention is about 1700 pg (sc)FcRn/mm² chip surface (corresponding to 1700 RU).

Second, by performing the SPR-chip coating in a pH-dependent manner controlled scFcRn monomer immobilization is achieved. Thus, the immobilization is carried out at pH 7.4 to ensure that that the scFcRn is immobilized in monomeric form and does not form dimers or multimers during the immobilization process. Thereby a low amount of scFcRn, i.e. about 80-120 RU, can be covalently conjugated to the solid surface. In contrast thereto, if the immobilization of the scFcRn is performed at pH dimeric scFcRn is immobilized due to heterodimerization occurring at this pH value.

In one specific embodiment, the sensor surface is an SPR chip with carboxymethylated surface, wherein the carboxyl groups are attached directly to the surface layer and which has no dextran matrix. In this embodiment a beta-2-microglobulin-human FcRn fusion polypeptide (the groups are linked by a (GGGGS)₄-peptidic linker) comprising a C-terminal His(10)-Avi-tag is immobilized to the solid surface using amine coupling at neutral pH (about 250 μg/ml in 10 mM HEPES at pH 7.4). Thereby about 80 RU of the FcRn are covalently conjugated to the solid surface. The employed running buffer is either 10 mM MES, 150 mM NaCl, pH 5.8, 0.05% P-20 or HBS-P buffer (10 mM HEPES, pH 7.4, 150 mM NaCl, 0.05% P-20).

Using the method according to the invention the effect of the Fab-FcRn-interaction as well as the Fc-region-FcRn-interaction can be analyzed. In FIG. 27 the 2-dimensional diagrams of five Fab-charge variants of the same parental anti-CD44 antibody are shown. It can be seen that, depending on the kind of modification, the Fab-FcRn interaction is changed. In FIG. 28 the 2-dimensional diagrams of four Fc-region variants of the same parent anti-CD44 antibody (upper left diagram) are shown. It can be seen that depending on the kind of modification the Fc-region-FcRn interaction is changed.

Thus, the effects delineated in the current invention can be shown using a 2- or 3-dimensional diagram, wherein the dissociation constant (stability; log kd) is shown on the x-axis and the association constant (recognition; (log ka) is shown on the y-axis. By using such a 2- or 3-dimensional diagram the influence of on the one hand Fc-engineering and on the other hand of the Fab-FcRn interaction on the overall antibody-FcRn interaction can be visualized. Especially the interrelation of Fc- and Fab-FcRn binding strength can be separated (see FIG. 17 and FIG. 18).

For the generation of such a 2- or 3-dimensional diagram any suitable software can be used, such as, e.g., the Interaction Map (IM) software of Ridgeview Diagnostics AB (Uppsala, Sweden).

By simplifying the antibody it is possible to monitor the effect of a single modification in the antibody on the overall antibody-FcRn-interaction (see FIG. 29 to FIG. 31).

The current invention is based, at least in part, on the finding that the pH-dependent overall antibody-FcRn interaction strength has to be in the range of 100-400 nM at pH 5.5-6.0 for selecting an antibody with suitable pH-dependent FcRn-mediated antibody recycling and thereby long in vivo half-life. The overall antibody-FcRn interaction strength comprises the Fc-FcRn- and the Fab-FcRn interaction.

A high one side Fc binding strength (e.g. 200 nM or higher) in the range between pH and pH 6.5 should be connected to a low or non-detectable binding strength at pH 7.4 and higher. This can be used for the selection of antibody variants with improved pharmacokinetic properties.

The fraction of the total interaction that results from Fab-FcRn-interaction can be derived from the additional spots visible in the 2- or 3-dimensional diagram and/or by analyzing the interaction of the Fc-region separately (e.g. after cleaving off the Fab-fragment). The higher the number of additional spots/the number of affected spots the more Fab-FcRn-interactions are present.

Said Fab-FcRn interactions can be reduced, e.g. by mutating residues in the Fab, or the pH-dependent Fc-region-FcRn-interaction can be increased, e.g. by Fc-engineering. At best, both engineering technologies are combined.

For example, the antibodies mAb-1, mAb-2 and mAb-3 in FIG. 31 and FIG. 32 are FcRn-binding silent in one Fc-region polypeptide by introducing the mutations I253A/H310A/H435A and have an increased FcRn affinity in the respective other Fc-region polypeptide by introducing the mutations M252Y/S254T/T256E and M252Y/S254T/T256E/T307Q/N434Y, respectively. It can be seen that the Fab-FcRn interaction is reduced and the Fc-region-FcRn affinity is increased. Thereby the antibody-FcRn interaction becomes dependent on the Fc-region-FcRn interaction solely and the contribution of/the distortion by the Fab-FcRn interaction is almost eliminated.

The upper border for improving/increasing the Fc-FcRn interaction is the complete elimination of pH-dependent binding. Such an elimination is, e.g., achieved by introducing the mutations MST-HN (Met252 to Tyr, Ser254 to Thr, Thr256 to Glu, His433 to Lys, and Asn434 to Phe) in a human IgG1 wt-Fc-region (see, e.g., Patel et al., J. Immunol. 187 (2011) 1015-1022).

Which kind of interaction between the Fab and the FcRn is present, i.e. charge- or hydrophobicity-based, can be determined using different buffer compositions in the SPR analysis. For example, the presence of a hydrophobic Fab-FcRn-interaction can be seen if the addition of ethylene glycol to the SPR buffer results in an increase in the Fc-FcRn/Fab-FcRn-interaction ratio. Likewise, the presence of an ionic/charge-driven Fab-FcRn-interaction can be seen if the addition of salt to the SPR buffer results in an increase in the Fc-FcRn/Fab-FcRn-interaction ratio (see FIG. 9 to FIG. 14, and FIG. 29 to FIG. 32).

This is summarized in FIG. 33.

With the method according to the current invention the different antibody-FcRn-interactions can be analyzed separately (see FIG. 34).

Based on the separation of the different interactions, the method according to the current invention can be used for multiple applications during antibody development and selection/screening.

One aspect is the method according to the current invention for antibody screening. In antibody screening, the affinity/strength of the Fc-FcRn-interaction is the selection criterion. The intramolecular avidity can be determined/weakened by the increase/addition of salt. The intermolecular avidity can be weakened by solution density. This is shown schematically in FIG. 35.

One aspect is the method according to the current invention for antibody engineering. In antibody engineering, the increase of the ratio between Fc-FcRn-interaction and Fab-FcRn-interaction is the target criterion. For example, different Fc-region engineering, i.e. the introduction of different FcRn-binding influencing mutations, result in different patters (see FIG. 36).

To show the effect of Fc-region engineering on antibody-FcRn-interaction the antibodies Briakinumab (Ozespa™) and Ustekinumab (Stelara™) were used as a model system. Both Briakinumab and Ustekinumab are fully human monoclonal IgG1 antibodies. They bind to the same human p40-subunit of interleukin 12 (IL-12) and interleukin 23 (IL-23) and they are not cross-reactive to the corresponding mouse IL-12 and IL-23. Briakinumab and Ustekinumab are an IgG1κ antibody with variable heavy and light chain domains of the VH5 and Vκ1D germline families and an IgG1 antibody with variable heavy and light chain domains of the VH3 and Vλ1 germline families, respectively. In addition to different variable domains, Briakinumab and Ustekinumab show differences in several allotype-specific amino acids in the constant domains (see alignment in FIG. 40 and FIG. 41; Sequence alignment of Briakinumab and Ustekinumab light and heavy chains—VH and VL regions are shown in italics; CDRs are marked with an asterisk (*)).

However, the amino acid differences are outside of the (cognate) FcRn binding regions and can therefore be considered to play no role in FcRn-dependent pharmacokinetics (see, e.g., Ropeenian, D. C. and Akilesh, S., Nat. Rev. Immunol. 7 (2007) 715-725). Interestingly, Ustekinumab has a (reported) median terminal half-life of 22 days (see Zhu, Y., et al., J. Clin. Pharmacol. 49 (2009) 162-175), whereas Briakinumab has a terminal half-life of only 8-9 days (see Gandhi, M., et al., Semin. Cutan. Med. Surg. 29 (2010) 48-52; Lima, X. T., et al. Expert. Opin. Biol. Ther. 9 (2009) 1107-1113; Weger, W., Br. J. Pharmacol. 160 (2010) 810-820).

The amino acid sequences of the antibody Briakinumab are reported in WO 2013/087911 (SEQ ID NO: 01 and SEQ ID NO: 02), of the antibody Ustekinumab in WO 2013/087911 (SEQ ID NO: 03 and SEQ ID NO: 04) and of the antibody Bevacizumab in Drug Bank entry DB00112.

The pH-dependent 2-dimensional diagram of the interaction of Ustekinumab with YTE mutation, which extends in vivo half-life, with FcRn is shown in FIG. 37. It can be seen that the interaction is strong at a low pH value and weaker at physiological pH value. This leads to efficient pH-dependent FcRn-mediated recycling and, thus, a long in vivo half-life.

The pH-dependent 2-dimensional diagram of the interaction of Briakinumab and Briakinumab with YTE mutation, which extends in vivo half-life, is shown in FIGS. 38 and 39, respectively. It can be seen that the interaction is strong at a low pH value well as at physiological pH value. This leads to impaired pH-dependent FcRn-mediated recycling and, thus, a short in vivo half-life. It can also be seen that Fc-region engineering in the case of Briakinumab leads to increased Fab-FcRn-interaction.

Additionally, a shift of the spots in the 2- or 3-dimensional diagram is an indication for Fc-region distortion resulting from the engineering of the wt-Fc-region.

Thus, by using a 2- or 3-dimensional visualization of the antibody-FcRn-interaction additional in vivo effects, e.g. compared to an FcRn column chromatography, can be analyzed.

### SUMMARY

Antibody half-life is mediated by FcRn. The underlying recycling mechanism is based on the pH dependent binding of antibodies to FcRn. It has been described previously that the antibody FcRn interaction is a two-pronged binding mechanism (Jensen et al., Mol. Cell Proteom. 16 (2017) 451-456). This mechanism was elucidated by Hydrogen-Deuterium exchange (HDX).

The antibody Fc-region comprises two heavy chains. All assay setups utilizing surface bound FcRn are hampered by the problem that the kinetic behavior is a mixture of 2:1 and 1:1 interactions. Depending on the applied FcRn coating density the Fc-region is able to interact with both or only one binding site.

The classical coupling chemistry usually only allows a random occupation of the SPR chip. Only the probability of a local high FcRn density can be directed by lower FcRn concentrations on the chip.

With the method according to the current invention it could be demonstrated that FcRn displays also a pH dependent self-interaction. This interaction has to be taken into account for an assay setup that allows more detailed information about the mechanistic details.

Thus, this aspect of the current invention is a novel SPR-based Fc-FcRn binding assay that accounts for individual interactions in Fc-FcRn binding assessment.

This aspect of the invention is based, at least in part, on the pH dependent FcRn coating on a solid phase, such as, e.g., an SPR-chip.

The method according to this aspect of the invention has been confirmed by reducing the complexity of the antibody down to the Fc-region alone with just one active FcRn binding site and thereafter adding back the additional domains of the molecule one after another.

Complex kinetics can be resolved using the software Interaction Map. This allowed for characterization and isolation of antibody-FcRn interactions occurring simultaneously.

By comparing the data obtained with the method according to the invention with the respective wild type antibody binding profile, the Human Epithelial Recycling Assay (HERA) and in human FcRn transgenic mice the predictiveness of the in vitro assay according to the invention for (the higher complexity of) antibody trafficking and recycling such as in vivo pharmacokinetics was shown.

It has been demonstrated that in case of an anti-Dig antibody the Fab-FcRn binding contribution is so strong that in vivo no clear difference can be observed to the symmetric YTE engineered antibody, in a human transgenic mouse model.

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 SPR sensorgram of different humanized/chimeric human IgG1 Fc-region comprising antibodies (exploratory and approved); sensorgrams were recorded under the same SPR conditions with the same concentration and same monomer concentration; the only difference is the antigen binding site.

FIG. 2 Three-dimensional diagram (with the stability (log kd) on the x-axis, the recognition (log ka) on the y-axis and intensity on the z-axis) of an isolated, Fab-less Fc-region-FcRn-interaction, i.e. a theoretical 1:1 interaction of an isolated antibody Fc-region with a single FcRn-binding site obtained by introducing the mutations I253A/H310A/H435A (numbering according to Kabat is used herein) in one Fc-region polypeptide.

FIG. 3 Two-dimensional diagram of the interaction of a full-length, monospecific anti-digoxygenin antibody with FcRn on an SPR solid surface.

FIG. 4 Sensorgram of a simple Fc-region-FcRn-interaction, i.e. a 1:1 interaction of an isolated, Fab-less antibody Fc-region with a single FcRn-binding site obtained by introducing the mutations I253A/H310A/H435A in one Fc-region polypeptide and maintaining the corresponding wild-type Fc-region polypeptide as the respective other Fc-region polypeptide at low FcRn immobilization levels with amine coupling.

FIG. 5 Two-dimensional diagram (with the stability (log kd) on the x-axis and the recognition (log ka) on the y-axis) of a simple Fc-region-FcRn-interaction, i.e. a 1:1 interaction of an isolated, Fab-less antibody Fc-region with a single FcRn-binding site obtained by introducing the mutations I253A/H310A/H435A in one Fc-region polypeptide and maintaining the corresponding wild-type Fc-region polypeptide as the respective other Fc-region polypeptide at low FcRn immobilization levels with amine coupling.

FIG. 6 Sensorgram of a complex IgG1 full length antibody-FcRn-interaction.

FIG. 7 Two-dimensional diagrams of a complex IgG1 full length antibody-FcRn-interaction at low FcRn immobilization levels using amine coupling.

FIG. 8 Two-dimensional diagrams of a complex IgG1 full length antibody-FcRn-interaction at high FcRn immobilization levels using biotin/avidin coupling.

FIG. 9 Effect of isolated Fab-FcRn-interaction of an anti-digoxygenin-Fabs added to the Fc-region with a single FcRn binding site (mutations I253A/H310A/H435A in one Fc-region polypeptide and wild-type in the respective other Fc-region polypeptide) determined at 150 mM sodium chloride.

FIG. 10 Effect of isolated Fab-FcRn-interaction of an anti-digoxygenin-Fabs added to the Fc-region with a single FcRn binding site (mutations I253A/H310A/H435A in one Fc-region polypeptide and wild-type in the respective other Fc-region polypeptide) determined at 400 mM sodium chloride.

FIG. 11 Effect of isolated Fab-FcRn-interaction of an anti-digoxygenin-Fabs added to the Fc-region with a single FcRn binding site (mutations I253A/H310A/H435A in one Fc-region polypeptide and wild-type in the respective other Fc-region polypeptide) determined at 400 mM sodium chloride and 20% (w/w) ethylene glycol (MW=62.07 g/mol).

FIG. 12 Effect of isolated Fab-FcRn-interaction of an anti-digoxygenin-Fabs added to a wild-type IgG1 Fc-region determined at 150 mM sodium chloride.

FIG. 13 Effect of isolated Fab-FcRn-interaction of an anti-digoxygenin-Fabs added to a wild-type IgG1 Fc-region determined at 400 mM sodium chloride.

FIG. 14 Effect of isolated Fab-FcRn-interaction of an anti-digoxygenin-Fabs added to a wild-type IgG1 Fc-region determined at 400 mM sodium chloride and 20% (w/w) ethylene glycol (MW=62.07 g/mol).

FIG. 15 Sketch depicting the different intramolecular Fab-FcRn and Fc-region-FcRn interactions.

FIG. 16 Sketch depicting intramolecular and intermolecular antibody FcRn-interactions.

FIG. 17 Sensorgram with separated interrelation of Fc- and Fab-FcRn binding strength can be separated.

FIG. 18 Two-dimensional diagram with separated interrelation of Fc- and Fab-FcRn binding strength.

FIG. 19 Biotin/avidin coupling density for immobilizing (sc)FcRn to the surface of the SPR chip.

FIG. 20 Amine coupling for immobilizing (sc)FcRn to the surface of the SPR chip for controlling the coating density down to low levels.

FIG. 21 Two-dimensional diagram showing the Fc- and Fab-FcRn binding strength of an anti-digoxygenin antibody with wild-type IgG1 Fc-region using a chip with about 1700 RU of (sc)FcRn captured by biotin/avidin coupling.

FIG. 22 Two-dimensional diagram showing the Fc- and Fab-FcRn binding strength of an anti-digoxygenin antibody with IgG1 Fc-region with symmetric M252Y/S254T/T256E mutations with about 1700 RU of (sc)FcRn captured by biotin/avidin coupling.

FIG. 23 Two-dimensional diagram showing the Fc- and Fab-FcRn binding strength of an anti-digoxygenin antibody with wild-type IgG1 Fc-region using a chip with about 80 RU of (sc)FcRn captured by biotin/avidin coupling.

FIG. 24 Two-dimensional diagram showing the Fc- and Fab-FcRn binding strength of an anti-digoxygenin antibody with IgG1 Fc-region with symmetric M252Y/S254T/T256E mutations with about 80 RU of (sc)FcRn captured by biotin/avidin coupling.

FIG. 25 Two-dimensional diagram showing the Fc- and Fab-FcRn binding strength of an anti-digoxygenin antibody with wild-type IgG1 Fc-region using a chip with about 80 RU of (sc)FcRn captured by amine coupling.

FIG. 26 Two-dimensional diagram showing the Fc- and Fab-FcRn binding strength of an anti-digoxygenin antibody with IgG1 Fc-region with symmetric M252Y/S254T/T256E mutations with about 80 RU of (sc)FcRn captured by amine coupling.

FIG. 27 Two-dimensional diagrams of the Fab-FcRn and Fc-region-FcRn interaction of the parent and five Fab-charge variants of the same parental anti-CD44 antibody.

FIG. 28 Two-dimensional diagrams of the Fab-FcRn and Fc-region-FcRn interaction of four Fc-region variants of the same parent anti-CD44 antibody (upper left diagram) are shown.

FIG. 29 Monitoring the effect of the introduction of a M252Y/S254T/T256E mutation, i.e. a single modification, in the antibody on the overall antibody-FcRn-interaction using a one-armed Fab-Fc-region fusion.

FIG. 30 Monitoring the effect of the introduction of a V308P/Y436H mutation, i.e. a single modification, in the antibody on the overall antibody-FcRn-interaction using a one-armed Fab-Fc-region fusion.

FIG. 31 Monitoring the effect of the introduction of an I253A/H310A/H435A mutation, i.e. a single modification, and the removal of the Fab (vs. mAb-2 on FIG. 29) in the antibody on the overall antibody-FcRn-interaction using a one-armed Fab-Fc-region fusion.

FIG. 32 Monitoring the effect of the introduction of a T307Q/N434A and further of a V308P/Y436H mutation, i.e. two single modifications, in the antibody on the overall antibody-FcRn-interaction using a one-armed Fab-Fc-region fusion.

FIG. 33 Delinearization of hydrophobic and charge driven antibody-FcRn interactions.

FIG. 34 Analysis of the different antibody-FcRn-interactions.

FIG. 35 Two-dimensional scheme showing the determination/weakening of the intramolecular avidity by the increase/addition of salt and the determination/weakening of the intermolecular avidity by solution density.

FIG. 36 Interaction spot pattern showing that matching the parental antibody interaction spot pattern is preferred over a shifted spot pattern for pharmacokinetic engineering of an antibody, e.g. when introducing the YTE mutations as the antibody might have a modified thermostability

FIG. 37 pH-dependent 2-dimensional diagram of the interaction of Ustekinumab with YTE mutation with FcRn.

FIG. 38 pH-dependent 2-dimensional diagram of the interaction of Briakinumab with FcRn.

FIG. 39 pH-dependent 2-dimensional diagram of the interaction of Briakinumab with YTE mutation with FcRn

FIG. 40 Light chain amino acid sequence alignment of Ustekinumab and Briakinumab; CDRs are marked with an asterisk (*).

FIG. 41 Heavy chain amino acid sequence alignment of Ustekinumab and Briakinumab; CDRs are marked with an asterisk (*).

FIG. 42 Sketches of the antibodies used in the examples.

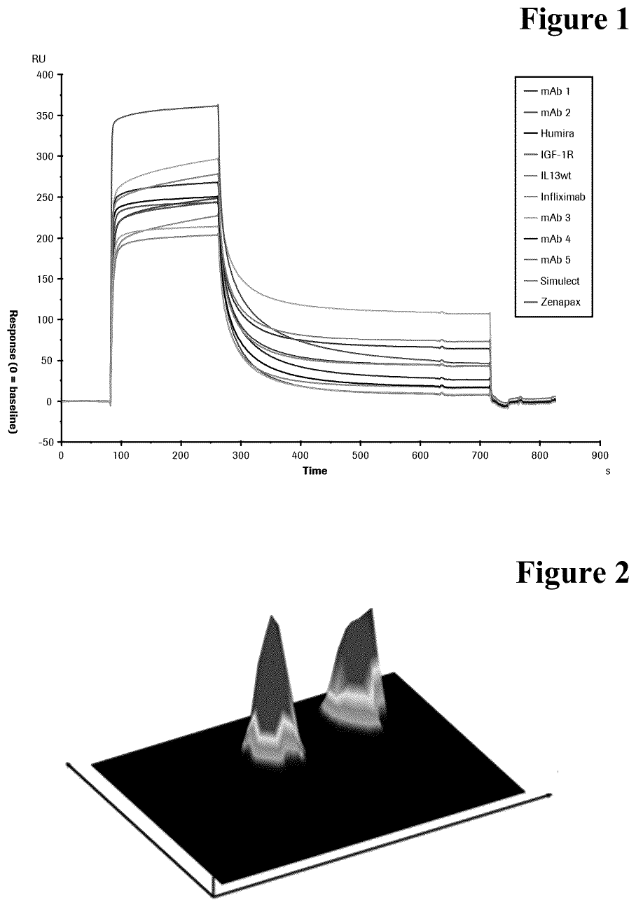

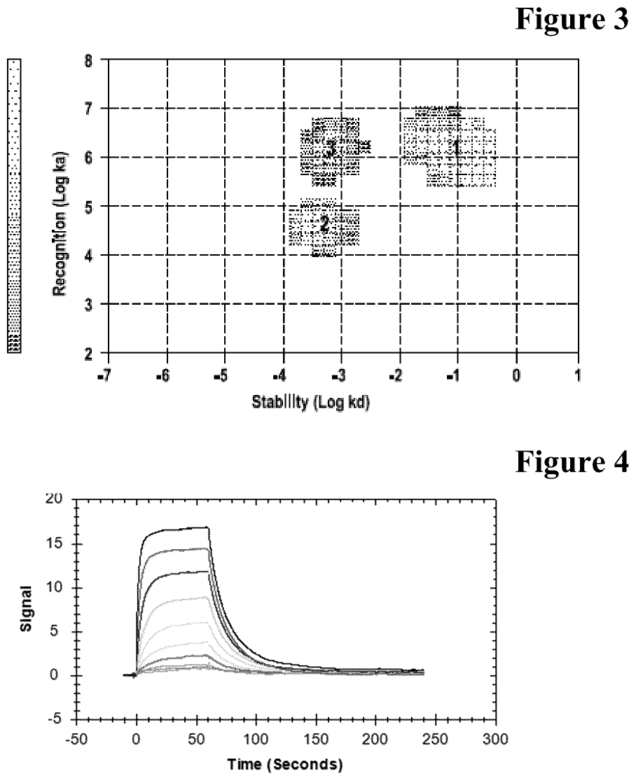

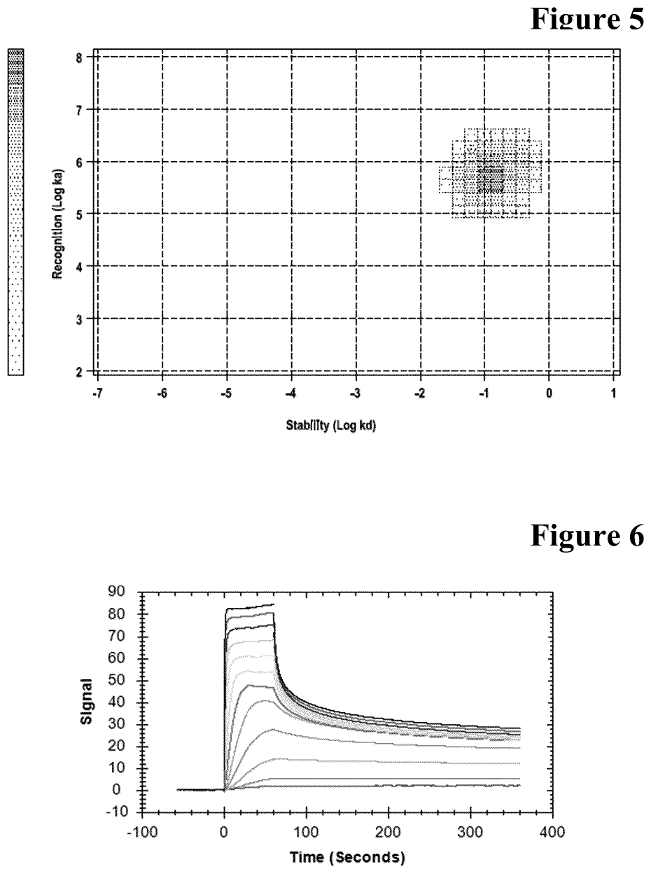

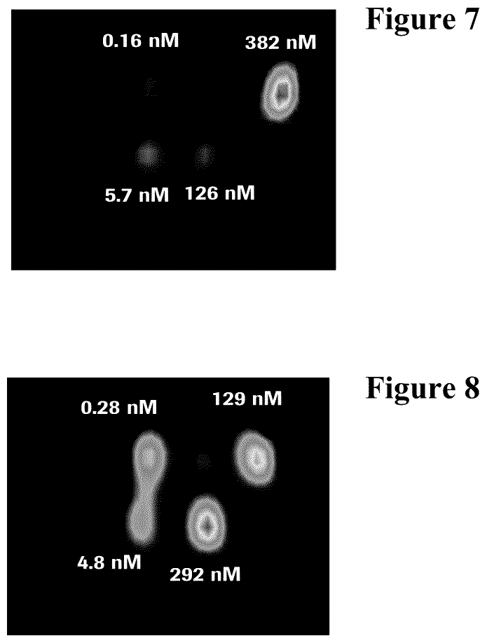

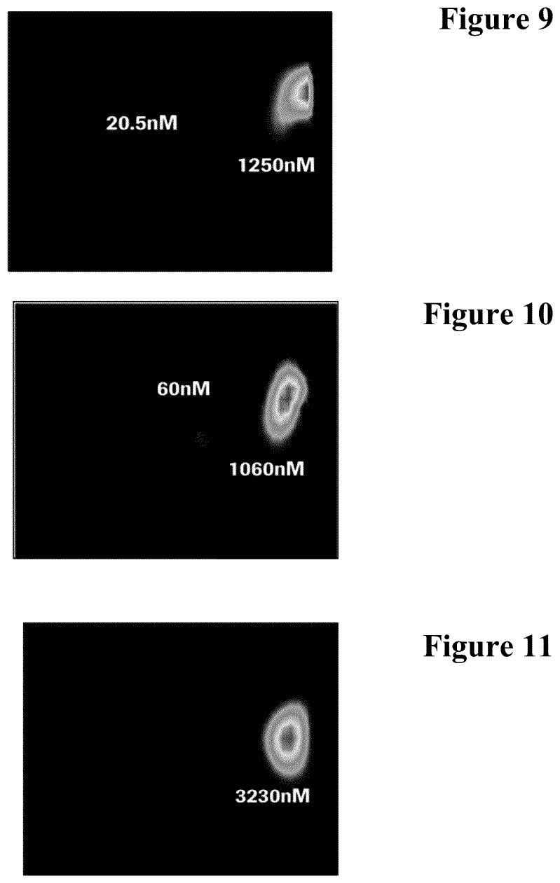

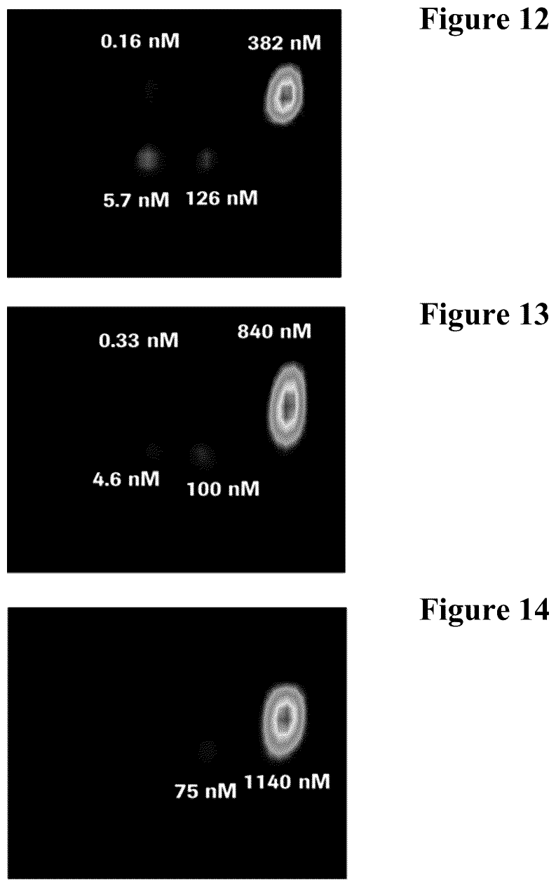

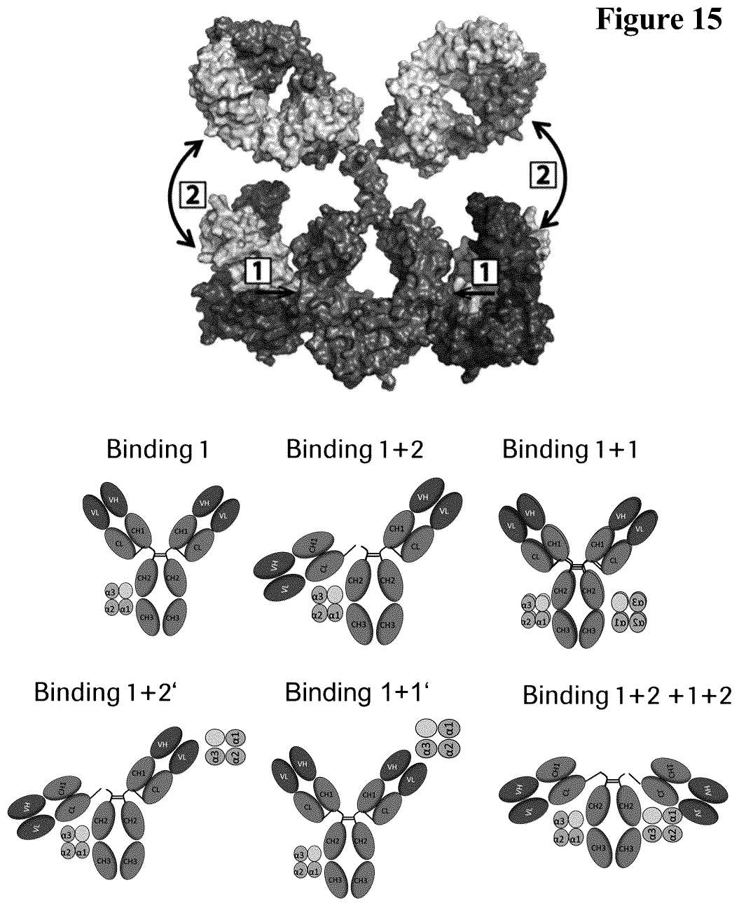

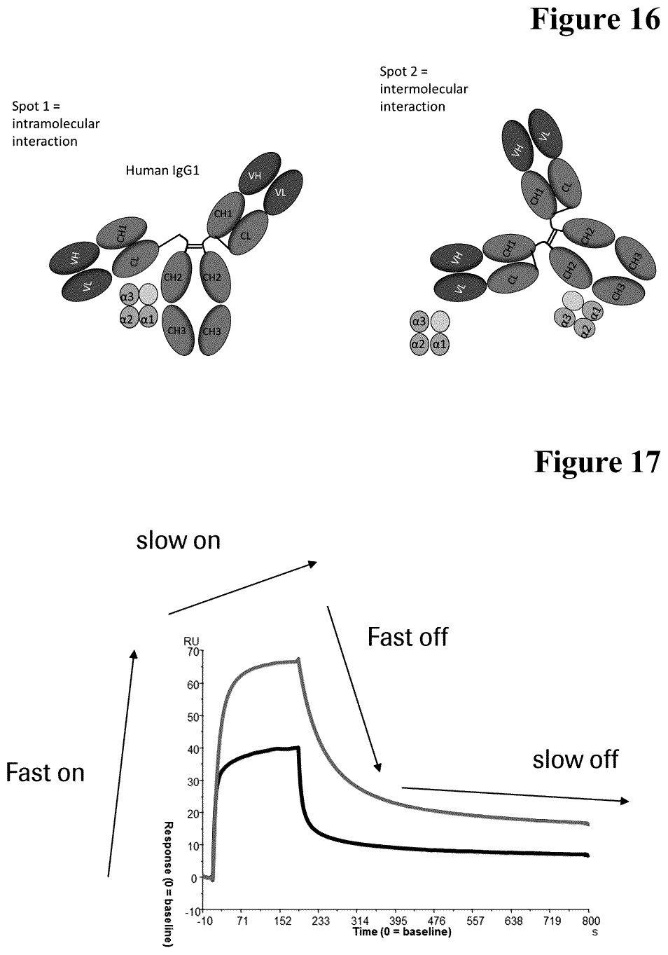

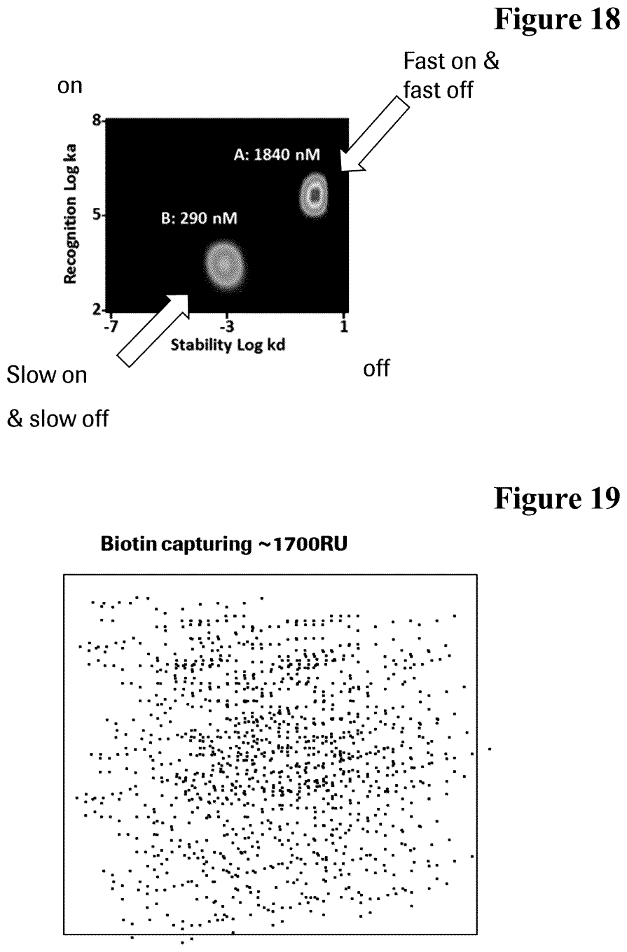

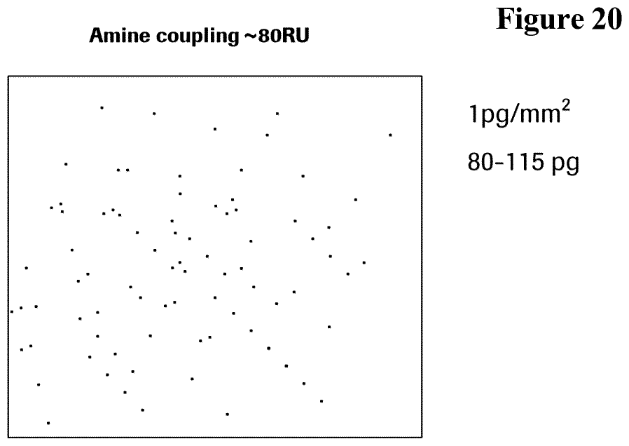

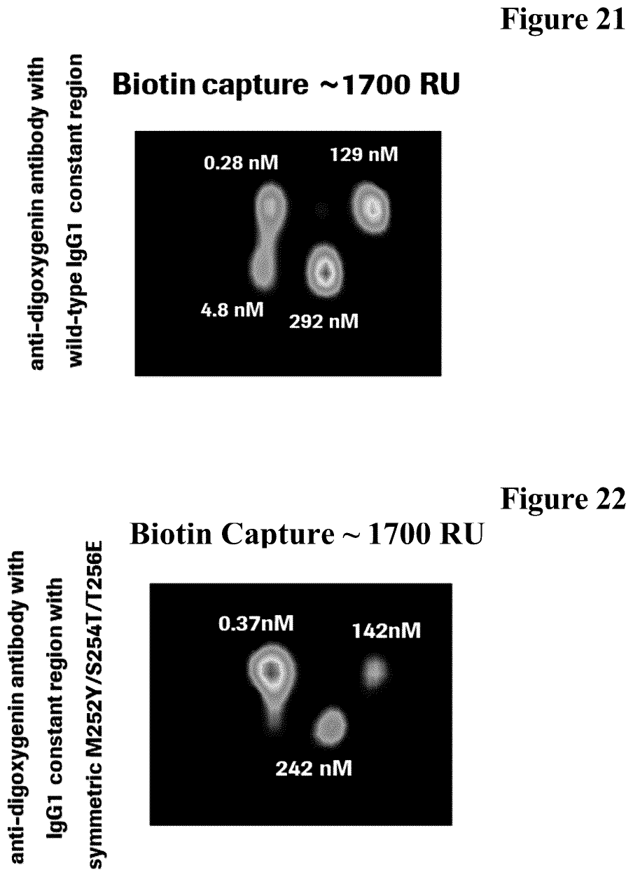

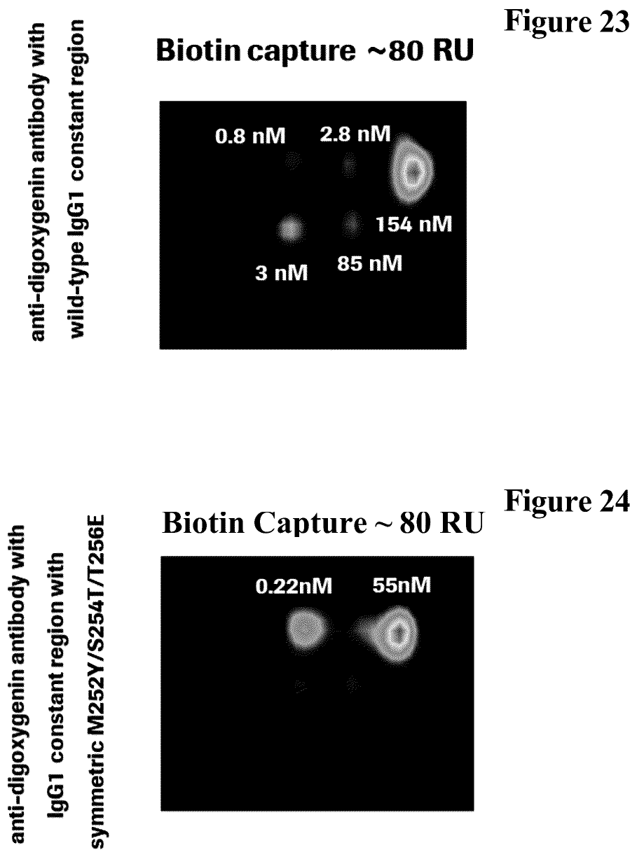

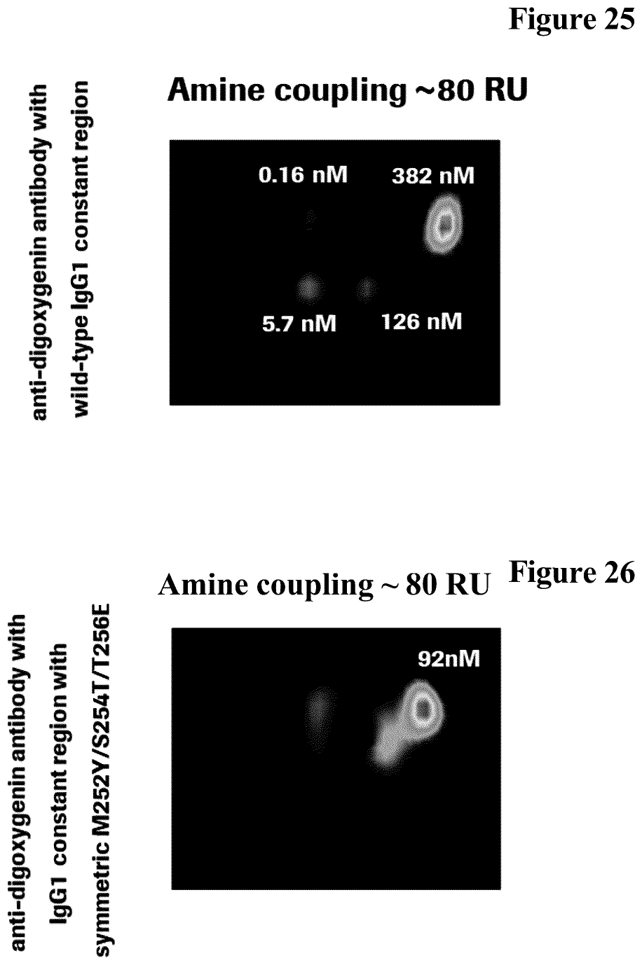

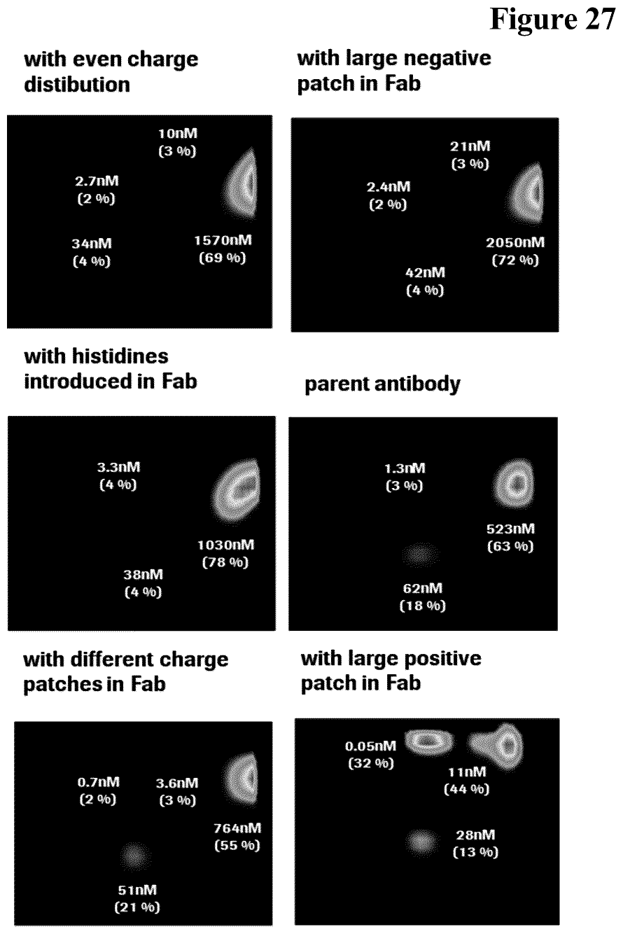

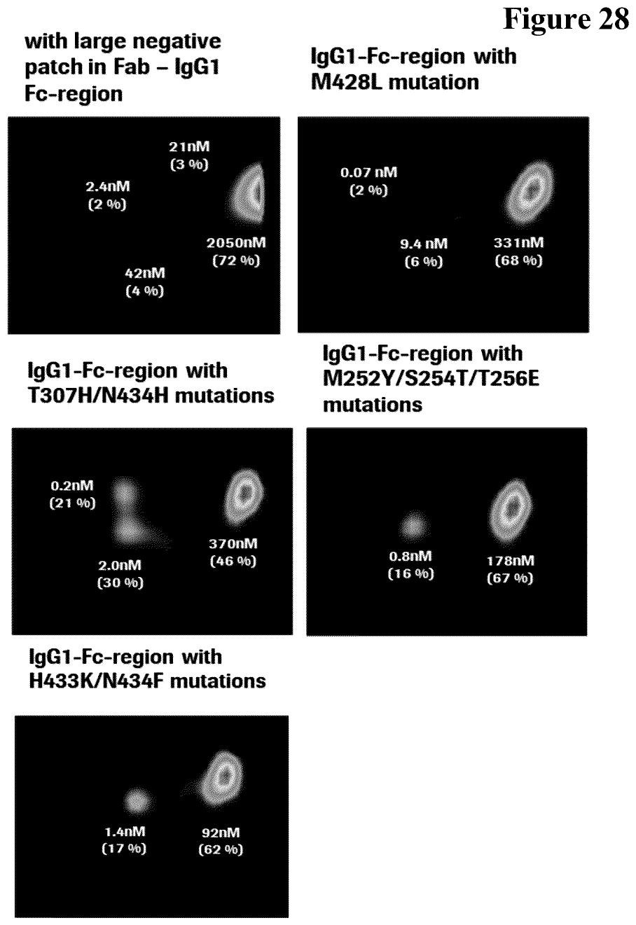

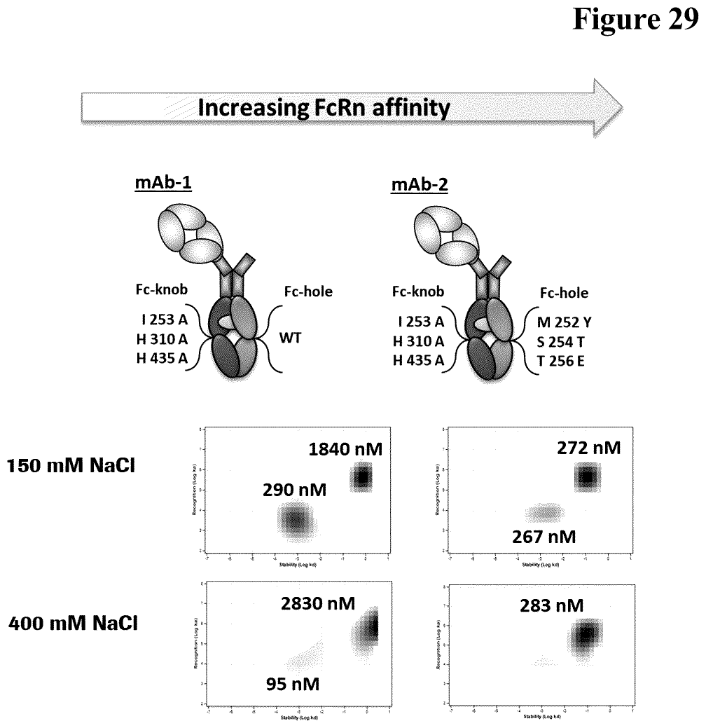

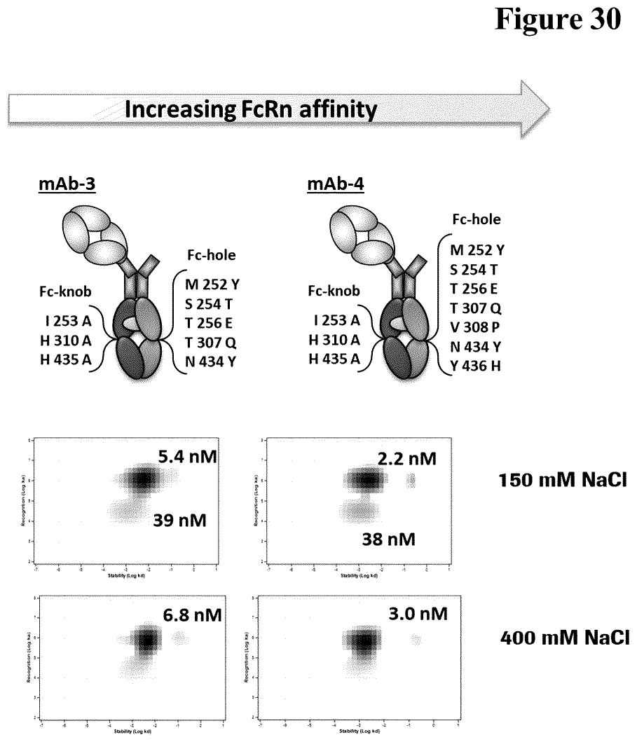

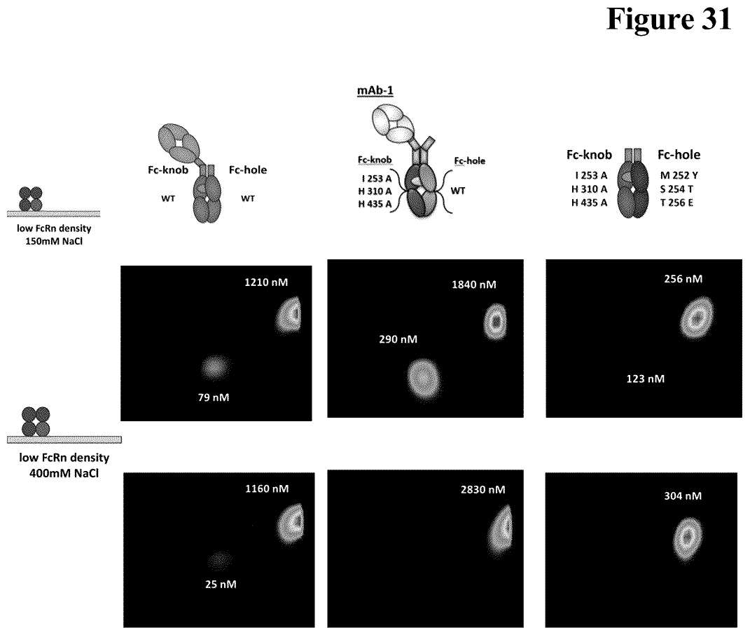

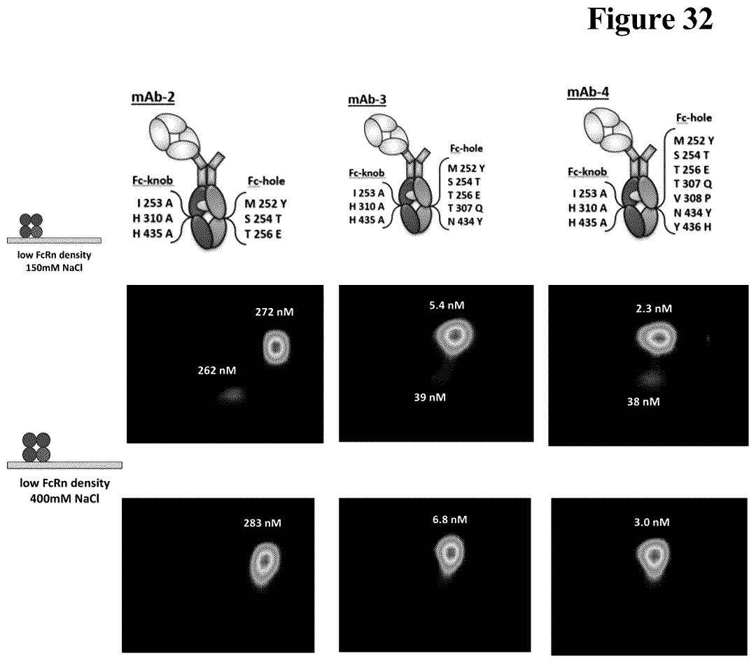

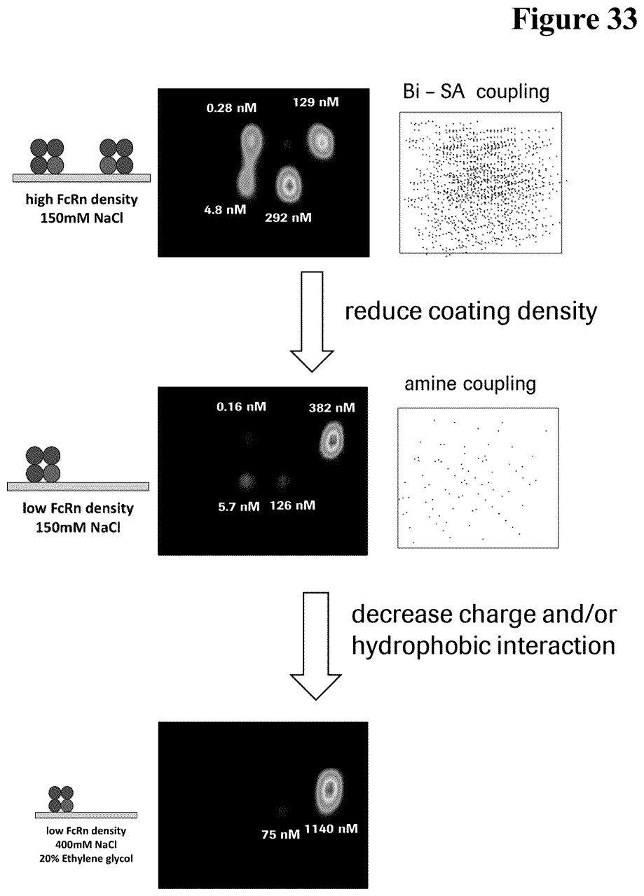

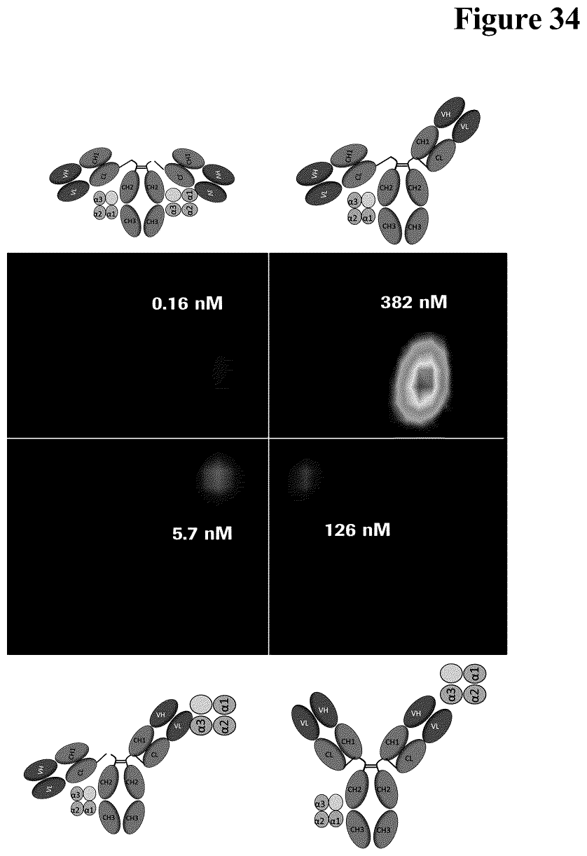

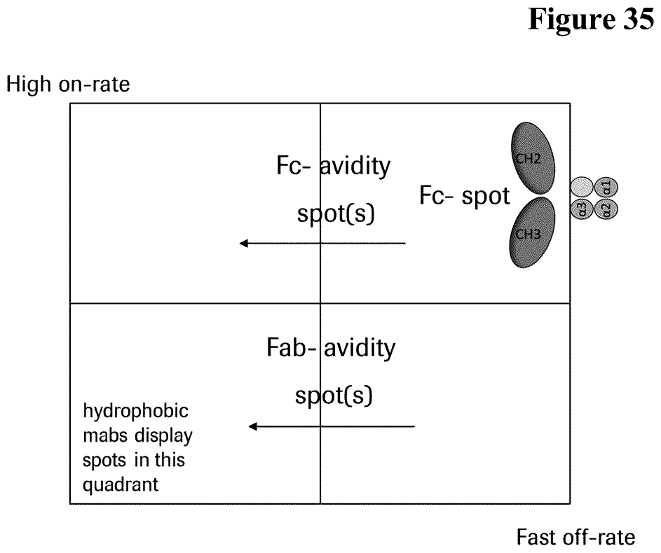

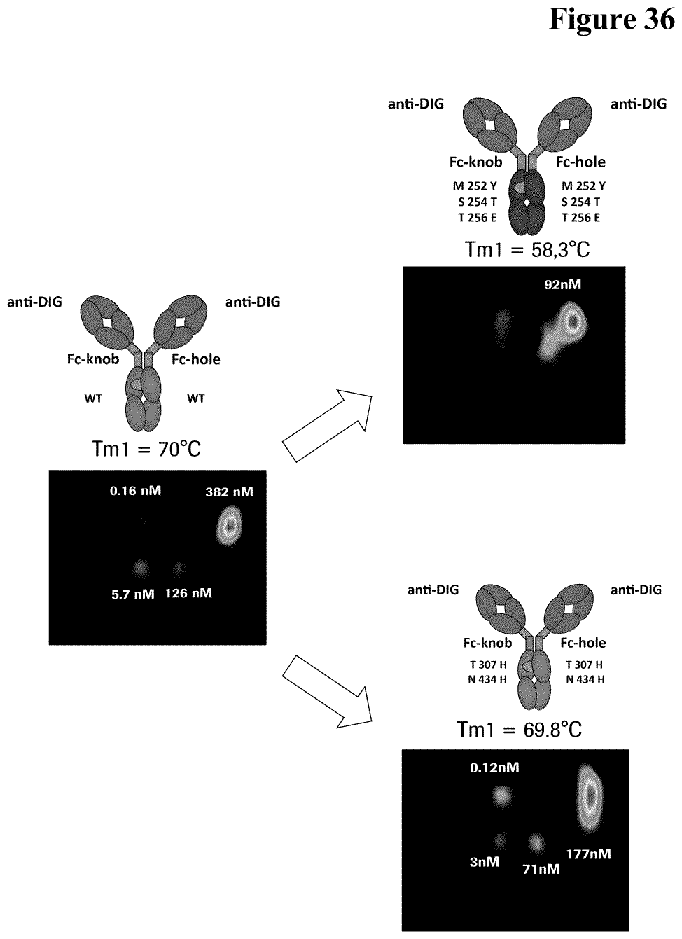

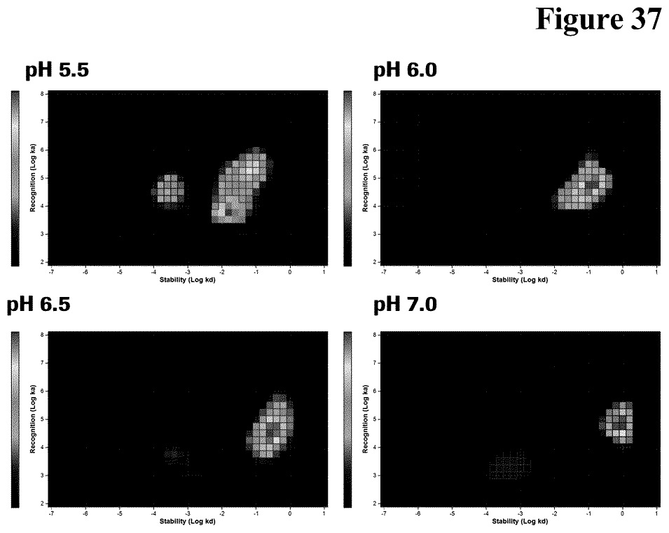

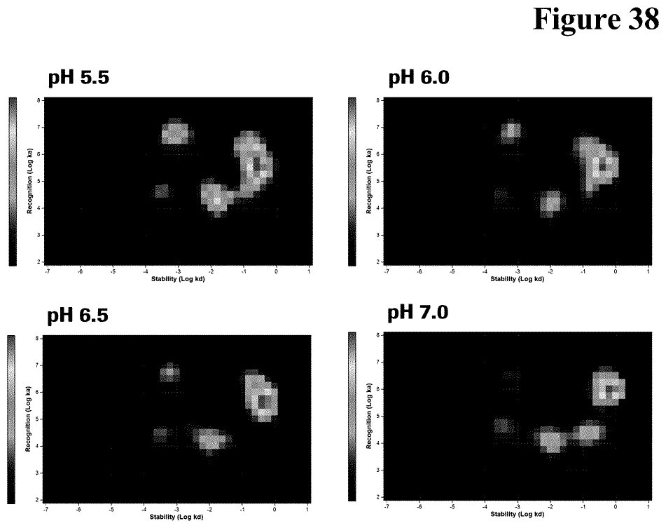

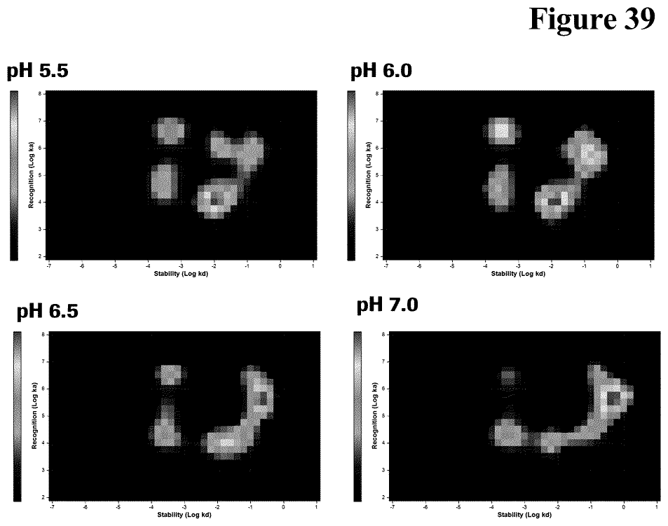

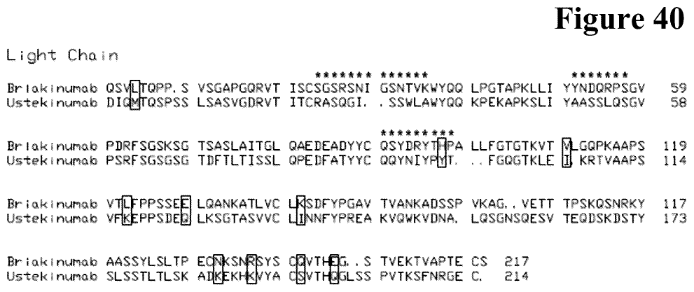

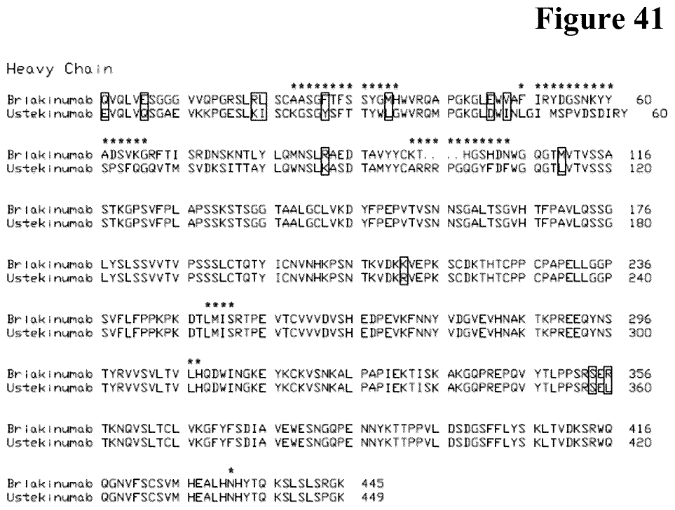

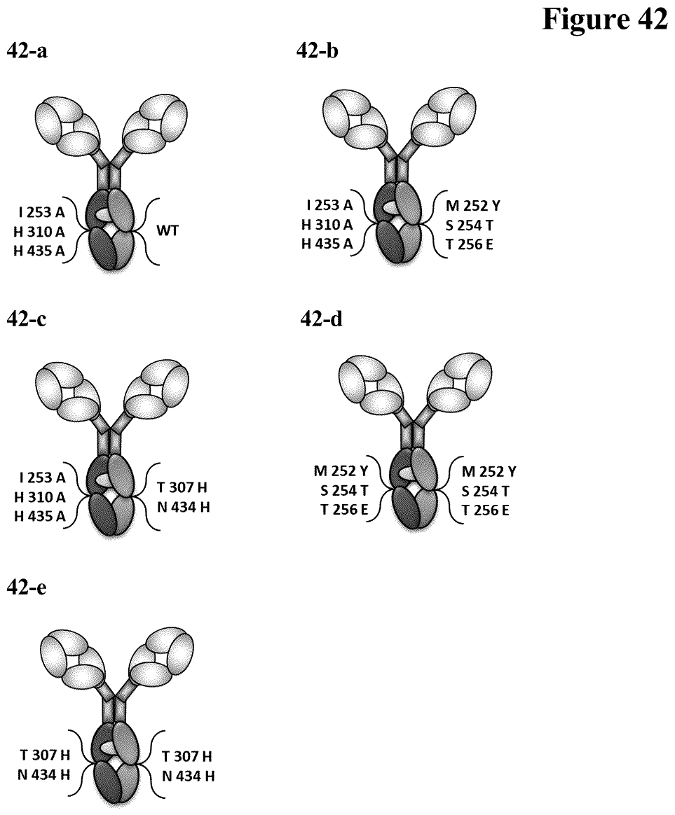

### MATERIALS

Information According to the Manufacturer:

Sensor chip C1 has a flat carboxymethylated surface. Provides the same functionality as Sensor chip CM5 but has no dextran matrix (the carboxyl groups are attached directly to the surface layer). The absence of a surface matrix makes Sensor chip C1 less hydrophilic than Sensor chip CM5. Experimental protocols follow the same principles for Sensor Chip C1 and Sensor chip CM5. The absence of a surface matrix will result in an immobilization yield that is approximately 10% of that obtained on Sensor chip CM5 under comparable conditions.

Amine coupling makes use of the N-terminus and c-amino groups of lysine residues of the ligand.

Immobilization Procedures

In general the immobilization procedure consists of three distinct parts:
Activation: the priming of the sensor chip so it can form a covalent bond with another molecule
Coupling: the injection of ligand so it forms covalent bonds with the sensor surface
Deactivation: injection of a low molecular reactive group to quench the remaining active surface groups

Activation:

In case of a covalent amine binding chemistry on the dextran-based sensor chips, the carboxyl groups are activated with a mixture of NHS (N-hydroxy succinimide) and EDC (1-Ethyl-3-(3-dimethylaminopropyl)carbodiimide) to create N-Hydroxy-succinimide esters. By varying the activation time, more or fewer carboxyl groups are activated. In addition, the concentration of the NHS/EDC mixture can be varied to control the quantity of activated carboxyl groups. The quantity of activated groups determines how much ligand can bind to the sensor surface. The standard activation period for a CM5 sensor chip of BIACORE is 7 minutes with 0.05 M NHS/0.2 M EDC at a flow rate of 5 μl/min.

Coupling:

The reactivity of the ligand at the chosen pH determines, how fast the ligand will bind to the activated surface. The rate of pre-concentration is directly related to the ligand concentration and pH of the immobilization solution. Ligand concentrations that are too high will give high ligand pre-concentration response but will also make it difficult to immobilize the proper amount of ligand. The relation between the amount of time the ligand is in contact with the activated surface and the amount of ligand bound is not linear as the sensor chip reaches saturation.

How much ligand to immobilize:

The amount of ligand to be immobilized depends on the application.

For specificity measurements, almost any ligand density will do as long as it gives a good signal.

Concentration measurements need the highest ligand density to facilitate mass transfer limitation. In a total mass transfer controlled experiment, binding will depend on the analyte concentration and not on the binding kinetics between the ligand and analyte.

Affinity ranking can be done with low to moderate density sensor chips. It is important that the analyte saturates the ligand within a proper time frame.

Kinetics should be done with the lowest ligand density that still gives a good response without being disturbed by secondary factors such as mass transfer or steric hindrance.

Low molecular mass binding should be done with high-density sensor chips to bind as much as possible of the analyte to gain proper signal.

In general, for kinetic measurements, a total analyte response of at most 100 RU, when the analyte is injected (1),(2) is desired (see mass transport). With this value in mind (Rmax), the amount of ligand (in response units) to be immobilized can be calculated with: Rmax response/Ligand response.

Deactivation:

The deactivation solution will block all remaining activated sites with an excess of reagent and because of its high ionic strength and high pH, the solution will wash away most of the electro-statically bound ligand. The amine coupling procedure is usually blocked with ethanolamine, but the use of BSA or casein is also possible. If high salt concentrations are detrimental to the ligand, the experimenter can just wait until all active sites are decayed back to carboxylic groups. The goal of the blocking is to remove the activated groups and make the surface as inert as possible.

In cases where positively charged analytes are being analyzed, the surface can be blocked with ethylenediamine to reduce the negative charge of the sensor surface and thus decrease the potential for non-specific binding.

### REFERENCES

(1) Karlsson, R. et al Kinetic analysis of monoclonal antibody-antigen interactions with a new biosensor based analytical system. Journal of Immunological Methods 229-240; (1991).
(2) Myszka, D. G. Survey of the 1998 optical biosensor literature. J. Mol. Recognit. 12: 390-408; (1999).

Amine Coupling

Amine coupling makes use of the N-terminus and c-amino groups of lysine residues of the ligand. The numbered points refer to the different stages in the immobilization procedure.
1) Baseline for the unmodified sensor chip surface with continuous flow (5 μl/min).
2) 35 μl injection of NHS/EDC to activate the surface by modification of the carboxymethyl groups to N-Hydroxy succinimide esters.
3) Baseline after activation. Activation of the surface has only a very slight effect on the SPR signal (100-200 RU).
4) Injection of ligand (10-200 μg/ml) leads to electrostatic attraction and coupling to the surface matrix. At this point, the ligand solution is still in contact with the sensor surface, and response includes both immobilized and non-covalently bound ligand. The N-Hydroxy succinimide esters react spontaneously with the amines on the ligand to form covalent links (1).
5) Immobilized ligand before deactivation. The ligand has passed the sensor surface and most of the protein that is not covalently bound is eluted.
6) Deactivation of unreacted NHS-esters using 35 μl of 1 M ethanolamine hydrochloride adjusted to pH 8.5 with NaOH. The increased SPR signal is due to a change in the bulk refractive index. The deactivation process also removes any remaining electrostatically bound ligand.
7) Point 7 minus Point 3 gives the amount of immobilized ligand after deactivation.

Amine coupling is the first choice with new molecules to couple. However acidic ligands (pI<3.5) are difficult to immobilize. Also when the free amine groups are in the biological active site, one of the other chemistries must be investigated.

### REFERENCES

(1) Johnsson, B. et al Immobilization of proteins to a carboxymethyldextran-modified gold surface for biospecific interaction analysis in surface plasmon resonance sensors. Analytical Biochemistry 198: 268-277; (1991).

### Example 1

General SPR-Method for Determining Fc-FcRn Interaction

All measurements were carried out at 25° C. using a BIAcore T200 instrument (GE Healthcare). Biotinylated single chain human FcRn was used for all interaction studies.

FcRn was immobilized in two different ways, a low density immobilization and a high density immobilization.

For the low density immobilization, FcRn was immobilized on a C1 chip using standard amine coupling. Therefore, the protein was diluted to a concentration of mg/ml with buffer (10 mM HEPES; pH 7.4) and injected for 60 sec. over the chip surface. The immobilization resulted in immobilization levels of around 80 RU.

For the high density immobilization, the FcRn was immobilized through biotin capturing. At first Neutravidin (ThermoScientific) was immobilized on a C1-chip using standard amine coupling. The Neutravidin was diluted in 10 mM Na-Acetate buffer at a pH of 4.5 to a concentration of 0.1 mg/ml and injected for 6 min. over the chip surface. The immobilization resulted in immobilization of around 1000 RU. Following the immobilization of Neutravidin, FcRn was captured by injecting the biotinylated protein for 5 min. over the chip with a concentration of 0.24 mg/ml. The capturing resulted in an immobilization level of around 1700 RU.

For the interaction measurements with different antibodies a buffer consisting of 10 mM MES at pH 5.8, 150 mM NaCl and 0.05% P-20 was used. The antibody-FcRn interactions were analyzed tested using single cycle or multi cycle kinetics and 2-fold or 3-fold dilution series. Recorded sensorgrams were double reference subtracted using a reference flow cell and blank injections. The resulting sensorgrams were evaluated using the TraceDrawer software (Ridgeview Instruments AB).

1. A method for determining antibody-FcRn-interaction comprising the steps of
a) immobilizing FcRn on a solid surface, which is suitable for surface plasmon resonance measurement,
b) individually applying to the solid surface obtained in step a) solutions comprising the antibody at different concentrations and determining the association rate constant and the dissociation rate constant for each concentration,
c) determining with the rates obtained in step b) the K_{D}-value of the antibody-FcRn-interaction,
wherein the immobilized FcRn is monomeric FcRn,
wherein the monomeric FcRn is immobilized using functional (capture) groups that are directly attached to said solid surface,
wherein the solid surface is free of branched glucan, and
wherein the immobilization of the FcRn is at a pH value of from pH 7 to pH 8.

2. The method according to claim 1, wherein the immobilization is at a pH value of about pH 7.4.

3. The method according to any one of claims 1 to 2, wherein the FcRn is immobilized at a density of 50-150 RU.

4. The method according to any one of claims 1 to 3, wherein the FcRn is a single chain FcRn (scFcRn).

5. The method according to claim 4, wherein the scFcRn is a fusion polypeptide of beta-2-microglobulin and human FcRn fusion polypeptide, which are conjugated to each other by a (GGGGS)₄-peptidic linker, and which comprises a C-terminal Avi-tag.

6. The method according to any one of claims 1 to 5, wherein the FcRn is immobilized using amine coupling or biotin/streptavidin coupling.

7. The method according to any one of claims 1 to 6, wherein the FcRn is immobilized at a density of about 50-150 pg/mm² chip surface.

8. The method according to any one of claims 1 to 7, wherein the immobilization is with a solution comprising FcRn at a concentration of about 250 μg/ml in 10 mM HEPES buffer at a pH value of pH 7.4.

9. The method according to any one of claims 1 to 8, wherein the solution of the antibody applied to the immobilized FcRn in step b) comprises 150 mM NaCl or 400 mM NaCl or 400 mM NaCl and 20% (w/w) ethylene glycol.

10. The method according to claim 9, wherein the solution of the antibody applied to the immobilized FcRn in step b) comprises either 10 mM MES, 150 or 400 mM NaCl, 0.05% P-20 and optionally 20% (w/w) ethylene glycol at pH value of pH 5.8, or comprises 10 mM HEPES, 150 mM or 400 mM NaCl, 0.05% P- and optionally 20% (w/w) ethylene glycol at a pH value of pH 7.4.

11. The method according to any one of claims 1 to 10, wherein the branched glucan is dextran.

12. The method according to any one of claims 1 to 11, wherein the Fab-FcRn interaction as well as the Fc-region-FcRn interaction are divided and visualized using a 2-/3-dimensional diagram, in which the stability (log kd, off-rate) is shown/corresponds to the x-axis and the recognition (log ka, on-rate) is shown/corresponds to the y-axis.


SEQUENCE LISTING (7 sequences)

SEQ ID NO: 1 - protein, 115 aa, synthetic construct [sequence omitted]

SEQ ID NO: 2 - protein, 112 aa, synthetic construct
  REGION 1..112: note = Amino acid sequence of the light chain variable region of briakinumab (J-695, ABT-874).
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG

SEQ ID NO: 3 - protein, 119 aa, synthetic construct
  REGION 1..119: note = Amino acid sequence of the heavy chain variable region of ustekinumab (CTNO-1275).
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 4 - protein, 107 aa, synthetic construct
  REGION 1..107: note = Amino acid sequence of the light chain variable region of ustekinumab (CTNO-1275).
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 5 - protein, 20 aa, synthetic construct
  REGION 1..20: note = G4S-linker
GGGGSGGGGSGGGGSGGGGS

SEQ ID NO: 6 - protein, 15 aa, synthetic construct
  REGION 1..15: note = Avi-Tag
GLNDIFEAQKIEWHE

SEQ ID NO: 7 - protein, 25 aa, synthetic construct
  REGION 1..25: note = His(10)-Avi-Tag
HHHHHHHHHHGLNDIFEAQKIEWHE